(19) United States
(12) Patent Application Publication
(10) Pub. No.: US 2024/0225480 A9
(43) Pub. Date: Jul. 11, 2024

(54) CANNULA FOR DETECTION OF THE VOLUME OF A CAVITY WITHIN A BODY AND CORRESPONDING ITEMS

(71) Applicant: RECO2VERY THERAPIES GMBH, Bad Klosterlausnitz (DE)

(72) Inventor: Torsten HEILMANN, Bad Klosterlausnitz (DE)

(21) Appl. No.: 18/546,846

(22) PCT Filed: Feb. 18, 2021
(86) PCT No.: PCT/EP2021/054011

§ 371 Date: Aug. 17, 2023

(51) Int. Cl. A61B 5/107; A61M 25/00

(52) U.S. Cl. CPC A61B 5/1073; A61B 5/1076; A61M 25/0026; A61B 2562/0247; A61B 2562/043

(57) ABSTRACT

Described is a cannula (110 to 1110, CA1500, CA1600) for detection of the volume (V) of a cavity within a body (100), comprising: —a lumen portion (LP) comprising and inner surface (ISF) and an outer surface (OSF), —a first opening (OP1) on a first end (FE) of the lumen portion (LP), —a second opening (OP2) on a second end (SE) of the lumen portion (LP), wherein the first opening (OP1) and the second opening (OP2) have an inner diameter that allows endovascular blood circuit support of the blood circuit of a subject (Pat), —a lumen (LU) extending from the first end (FE) to the second end (SE) within the lumen portion (LP), and —at least one series (SER1) of electrodes (EE1 to EE2) comprising at least two electrodes (EE1 to EE2), wherein the series of electrodes (EE1 to EE2) is arranged along at least a part of the lumen portion (LP) at the outer surface (OSF), wherein the at least one series (SER1) of electrodes (EE1 to EE2) is configured to be used for the detection of the amount of a volume (V) of a cavity within a body (100) of the subject (Pat).

Measuring of volumes and/or of pressure in cavities of a living body may be challenging, e.g. within the cavities of a human heart which beats for instance about 50 times per minute. Measuring of volumes and/or of pressures may be made more complicated by the flow of flowing fluids and/or by medical instruments which are arranged within the same cavity, e.g. catheters and/or cannula for blood transfer, etc.

Endovascular catheters/cannulas may be advanced in vessels, for instance plastic tubes or plastic tubes that are armed with metal. An incision may be made into the skin of a patient. The incision may have a length that is less than 5 cm (centimeter), less than 3 cm or less than 1 cm. Local anesthesia may be used thereby. A catheter may be used to insert a guide wire and. Dilators may be used to expand the incision and/or an opening within the vessel. A further catheter or a cannula for blood (or other liquid) transfer may be inserted into the vessel using the guide wire and/or an introducing member.

It is an object of the invention to provide an improved cannula, e.g. an endovascular cannula. Preferably, the cannula shall enable detection of volume and/or of pressure in a cavity of a living body. Furthermore, corresponding items shall be provided, preferably enabling advanced diagnostic and/or therapeutic treatment methods. Moreover, corresponding medical methods shall be provided.

### SUMMARY OF THE INVENTION

Cannula for endovascular blood circuit support and for detection of the volume of a cavity within a body, comprising:
a lumen portion comprising and inner surface and an outer surface,
a first opening on a first end of the lumen portion,
a second opening on a second end of the lumen portion,
   wherein the first opening and the second opening have an inner diameter that allows e.g. endovascular blood circuit support of the blood circuit of a subject,
a lumen extending from the first end to the second end within the lumen portion, and
at least one series of electrodes comprising at least two electrodes,
   wherein the series of electrodes is arranged along at least a part of the lumen portion at the outer surface,
   wherein the at least one series of electrodes is configured to be used for the detection of the amount of a volume of a cavity within a body of the subject.

The cannula may be configured to be used endovascularly, e.g. having an outer diameter of less than 33 French (1 French equal to 0.3 mm (millimeter)). Furthermore, the cannula may be configured to be connected to an extracorporeal blood pump, e.g. to a blood pump providing more than 1 l/min (liter per minute), more than 2 l/min, more than 4 l/min. However, the pump volume of the blood pump may be less than 6 l/min or less than 5.5 l/min.

The electrodes may be configured to determine and/or to measure the volume in a cavity of a living body.

The basic principle for the detection of the volume may be based on detecting the electrical conductivity or electrical resistivity of a body liquid, e.g. blood, or of an auxiliary liquid. The electrical conductance around the electrodes may depend on the volume of the cavity. Source electrodes may be used to generate an appropriate electrical field for measuring the conductance or resistivity of the liquid. The electrodes may have connections to the outside of the cannula, e.g. to the proximal end of the cannula. The connections may be made by wire(s) or wireless.

Between adjacent electrodes, there may be a respective electrically insulating region. Detection may refer to non-SI-units (SI System International) or only to values. Measurement may refer to measuring values according to SI-units (SI System International). The electrodes may be arranged circumferentially around the cannula. This may allow volume detection independent of the rotation angle of the cannula. The electrodes may be arranged equidistantly in order to enable the usage of simple calculations for the volume. The electrodes may be used to determine the volume of a chamber within the heart, e.g. of a ventricle or of an atrium or of another cavity of the heart.

The cannula may have only one wall with an inner surface and an outer surface. Alternatively, the cannula may have an inner wall and an outer wall. Electrodes and/or wires and/or electronic elements may be arranged between the inner wall and the outer wall. The wall(s) may be very flexible in order to allow endovascular insertion of the cannula, e.g. around curves and/or corners.

Alternatively or additionally, at least one other sensor may be integrated as well into the cannula, e.g. pressure sensor(s), blood conductivity sensor(s), ECG (Electrocardiography) sensor(s). Thus, a multifunctional cannula may be provided. Combining different types of sensor signals may allow advanced diagnostic features, e.g. PV (pressure-volume) loops which give much more information than pressure or volume alone.

In the following the longitudinal axis of lumen portion or the extension thereof beyond the lumen portion may be used as a reference axis. The terms “radial”, “axial” and/or “angularly” may be used with regard to this reference axis, similarly to cylinder coordinates which are used in a cylindrical coordinate system. In this application document the definition for “distal” is far from a person that inserts the cannula/catheter. “Proximal” means near to the person that inserts the cannula.

The lumen portion of cannula which is used for blood transfer may be the portion of the cannula that is insertable into a body of a subject, preferably endovascular, e.g. without surgery and mainly by using needles, dilators, guide wires, cannulas and/or introducer members. Alternatively, the lumen portion may be a portion which extends over the overall length of the cannula.

The length of the cannula/lumen portion from the first end to the second end may be at least 50 centimeters or at least 70 centimeters at least 90 centimeters. The outer diameter of the lumen portion may be at least 10 French (1 French equal to 0.3 mm (millimeter)) or at least 15 French or at least 20 French or at least 25 French or at least 30 French. Although catheters for volume measurement are known for decades, there was no proposal to combine a cannula for blood transfer with electrodes known from such catheters. Surprisingly, this combination enables advanced diagnostic and therapeutic treatments as is described below in more detail. The inner diameter of the cannula may be at most 1 French or at most 2 French smaller than the outer diameter. The outer diameter of the cannula may be less than 33 French. The length of the cannula may be less than 2 meters or less than 1 meter.

The number of electrodes may be in the range of 5 to 15 electrodes or in the range of 6 to 20 electrodes or more than 20. The number of electrodes may be less than 100. A conductance cannula for a ventricle of a heart may have for instance 7 to 9 or 6 to 10 electrodes. Further electrodes may be provided, for instance for volume measurements within one or within both atria of the heart. Alternatively or additionally, electrodes may be provided in order to determine the volume of blood vessels, for instance of the aorta. This may be useful for the treatment of specific diseases of the aorta, e.g. aneurysm. Thus, there may be more than one group (series) of electrodes.

The cannula may comprise at least one pressure sensor or at least two pressure sensors or at least three pressure sensors or at least four pressure sensors. The cannula may have no electrodes in this case. Alternatively, pressure sensors may be provided in addition to electrodes. The pressure sensor or the pressure sensors may be configured to detect the pressure within a cavity of the body. The pressure sensor may have a connection to the outside of the cannula, e.g. to the proximal end, by wire(s) or by a wireless connection. A piezoresistive pressure sensor may be used. Alternatively, piezoelectrical sensors or sensors which operate according to other physical principles. Sensors produced by Millar Instruments Inc. (may be a trademark) may be used for instance.

The cannula may comprise a second series of the at least one series of electrodes. The first series may be arranged closer to the second end (e.g. distal end) of the cannula than the second series. There may be a group distance between a last electrode of the first series which is farthest away from the second end and a first electrode of the second series which is closest to the second end. The group distance may be greater than or greater than twice or greater than the threefold of the greatest distance between two adjacent electrodes of the first series and/or greater than the greatest distance between two adjacent electrodes of the second series. The distances may be measured from the end of the last electrode to the beginning of the first electrode. Alternatively, measurement may be made from a center point/line of the last electrode in axial direction of the cannula to a center point/line of the first electrode in axial direction of the cannula. Other measurement points are possible as well, for instance usage of distal edges or of proximal edges. Thus, the comparably large group distance may be used when the cavities have a greater distance to one another or when the cannula is kinked or meanders or because of another reason. As an example, the electrodes of the first series may be configured to determine the volume of the left ventricle of a heart and the second series of electrodes may be configured to determine the volume of the left atrium of the heart.

Alternatively, the group distance between the last electrode of the first series which is farthest away from the second end and the first electrode of the second series which is closest to the second end may be less than the threefold or less than twice the greatest distance or less than the greatest distance between two adjacent electrodes of the first series and greater than the greatest distance between two adjacent electrodes of the second series. Again the same measurement schemes may apply. Thus, the comparably small group distance may be used when the cavities have a smaller distance to one another or when the cavities are adjacent to one another with a separating wall therein between or without such a separating wall. Exemplary, the electrodes of the first series of electrodes may be configured to determine the volume of the right ventricle of a heart and the second series of electrodes may configured to determine the volume of the right atrium of the heart. The same may apply to electrodes within the left atrium and the right atrium.

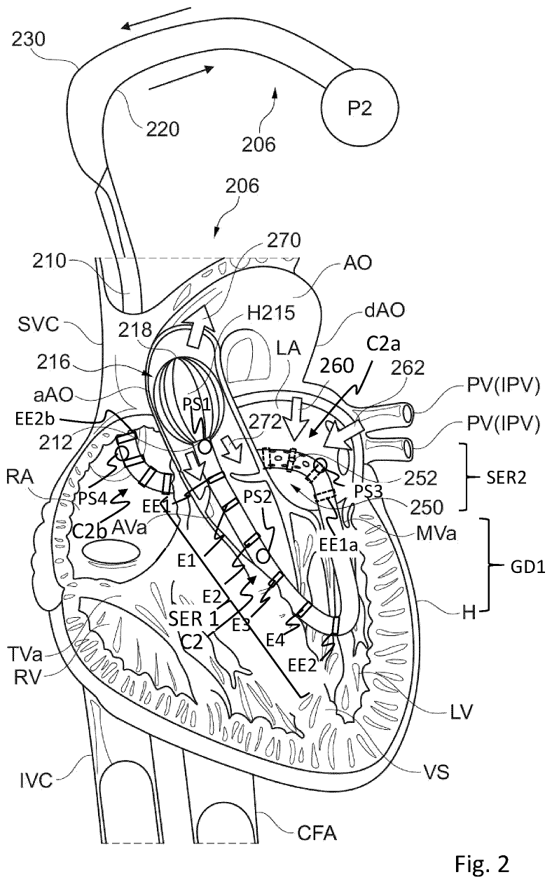

In an embodiment which is not claimed yet, there are at least three groups or series of electrodes arranged on the same catheter, e.g. on the two lumens of a dual lumen cannula or of a dual lumen cannula system. A first group distance between the first series and the second series of electrodes may be greater than a second group distance between the second series and the third series of electrodes. The first group distances may be as mentioned above for the first alternative of the length of the group distance and the second group distance may be as mentioned above for the second alternative of the length of the group distance, e.g. less than the threefold or less than twice the greatest distance or less than the greatest distance between two adjacent electrodes in the second series. This embodiment is described in more detail with regard to e.g. FIGS. 2, 3 and 10.

There may be at least one radial hole or there may be at least two radial holes or at least three radial holes between the electrodes of the at least one series of electrodes. The radials holes may not extend or may extend through the electrodes. The radial holes may be used as inlet holes or as outlet holes of the cannula. Thus, there may be a combination of measurement electrodes and radial fluidic holes within the same portion of the cannula allowing multiple functions.

The cannula may be a single lumen cannula which may be constructed in a simple manner. A single lumen cannula may provide enough space for wiring of the electrodes and/or for other electronic circuitry used for volume measurement and/or pressure measurement.

Alternatively, the cannula may be a bidirectional cannula comprising at least one third opening between the first opening and the second opening. The bidirectional cannula may comprise at least one valve arrangement which is configured to enable flows between different openings of the cannula depending on the direction of the flow within a common flow portion of the cannula. There may be a proximal first opening, a distal second opening and an intermediate portion with an intermediate opening. The portion between the proximal first opening and the intermediate opening may be the common fluid portion through which each fluid flow has to flow. In a first variant, the intermediate portion may be an outflow portion where fluid flows out of the bidirectional cannula. In the first variant, the distal second opening may be an inflow portion. In a second variant, the intermediate portion may be an inflow portion where fluid flows into the bidirectional cannula. In the second variant, the distal second opening may be an outflow portion. Bidirectional cannulas are produced for instance by the company PulsCath BV (may be a trademark).

The bidirectional cannula may not only be modified by pressure sensor(s) and/or electrodes for volume determination but also by an outer cannula in which the bidirectional cannula is inserted in order to guide flow from the intermediate opening to other relevant parts of the body which are farther away from the intermediate opening. Alternatively, the outer cannula may be used to guide flow to the intermediate opening from relevant parts of the body which are farther away from the intermediate opening.

A membrane pump (e.g. using an IABP (Intra-Aortic Balloon Pump) control module) may be used to pump fluid into and out of the bidirectional cannula, preferably at the first opening. Alternatively, a continuous pump may be used which may switch rotating directions. Furthermore, it is possible to use arrangements of more than one pump, e.g. membrane pump, in order to pump blood into and out of a single bidirectional cannula.

The cannula may be an inner cannula of a dual lumen cannula. The dual lumen cannula may comprise an outer cannula. The inner cannula may be arranged within the outer cannula and may extend more distally than the outer cannula. The inner cannula may be axially movable within the outer cannula or may be axially fixed arranged within the outer cannula. The dual cannula or a dual cannula system may allow advanced medical applications compared to the usage of only single lumen cannulas. Volume measurement and/or pressure measurement may be important for such advanced medical applications.

Alternatively, the cannula may be an outer cannula of a dual lumen cannula. The dual lumen cannula may comprise an inner cannula arranged within the outer cannula and extending more proximally than the outer cannula. The inner cannula may be axially movable within the outer cannula or may be axially fixed arranged within the outer cannula. Again, the dual cannula or a dual cannula system may allow advanced medical applications compared to the usage of only single lumen cannulas. Further, advantages may result from the usage of the electrodes for volume sensors and/or from the usage of pressure sensors.

The inner cannula may be axially movable with regard to the outer cannula, preferably along a distance which is at least half the length, the length or more than the length of the outer cannula. This axially movable (insertable) inner cannula may reduce trauma during insertion of both cannulas because the outer cannula is inserted first. The outer cannula may not be as stiff as the combination of the outer cannula and of the inner cannula. Furthermore, an increase of the stiffness because of wires for contacting the electrodes and/or pressure sensors may be acceptable as it is compensated by mentioned reduction of trauma up to a specific degree.

The cannula may comprise at least one volume expandable arrangement. The volume expandable arrangement may define a first volume in a non-expanded state and a second volume in an expanded state. The second volume may be greater than the first volume by at least factor 2, at least factor 3 or by at least factor 4 or more than factor 10. The factor may be less than 100. Thus, volume of the volume expandable arrangement may be variable. The defined volume may correspond to an enclosed volume and/or to an enveloped volume. The volume that is defined by a volume expandable arrangement may be an inner volume of the expandable arrangement, e.g. volume that is embraced or encompassed by the expandable arrangement.

The volume expandable arrangement may be a balloon which is pumped up for instance via a line for fluid, e.g. gas or liquid. Alternatively, a cage arrangement may be used. An introducer may be used to hold the cage in the collapsed state. If the introducer is pulled back out of the cannula the cage arrangement may automatically expand.

The volume expandable arrangement may be positioned at the end or at an intermediate portion of the cannula, for instance in order to prevent that tissue is sucked into holes of the cannula or that tissue is damaged by outflowing liquid. The volume expandable arrangement may fix (for some time) a distal end of cannula or another portion of the cannula within the body or within a body cavity. Other fixing means may be used as well, e.g. hooks.

The cannula may comprise at least one cage arrangement and/or at least one membrane, arranged at at least one wire of the cage arrangement. Nitinol (may be a trademark) wires may be used for the wires of the cage. Thermal effects of the material may be employed, for instance with regard to a form shape “memory”. Thus, metal wires may be used and modification of the electrical field by the wires may be considered, if any. Alternatively, non-conducting or electrically isolating wires may be used in order to not or only slightly influence the measurement of volume within the cavity. However, the volume expandable arrangement may be arranged a distance away from the electrodes such that the influence of volume expandable arrangement may be neglected during detection or measuring of the volume.

The membrane may be arranged around the cage, e.g. at least partially around the cage. In the collapsed state, the membrane may be folded or more folded than in the expanded state. In the expanded state of the cage arrangement the membrane may be without folds or pleats or with less folds than in the collapsed state. Folds may be used in the expanded state in order to realize a valve function of the membrane, i.e. fluid flow presses the membrane against adjacent issue in one direction and fluid flow in the other direction does not have such an effect but may be pass between membrane and tissue. The membrane may be made of electrically isolating material, e.g. non-conductive material. Thus, the membrane does not significantly influence measurement of volume using the electrodes and/or of pressure.

The cannula may comprise a flexible material extending from the first end to the second end. The material of the cannula/catheter may be biocompatible and/or comprise or consist of urethane, e.g. polyurethane and/or silicone and/or polyvinyl chloride. Silicone may be used as well, e.g. using dipping technologies and subsequent curing or hardening. The material may have a thickness of less than 1 mm (3 French) or of less than 0.5 mm (1.5 French). The material may form back into a released form or into a more released form after kinking, e.g. automatically. Resilient materials may be used as well. Metal cores may be used, e.g. spirally wound metal.

A further aspect relates to a set comprising a cannula according to one of the embodiments mentioned above and an extracorporeal pump. Thus, both components may be tuned to each other in order to enable better medical treatment. Endovascular insertion of the cannula avoids complicated surgical operations, e.g. the thorax has not to be opened. The pump may be a membrane pump, a centrifugal pump, a peristaltic pump or a pump of another type. Radial, axial or diagonal pumps may be used. Membrane pumps may be preferred because shear stress to blood is low for these pumps. Shear stress may destroy blood cells which are important for oxygen transport and/or for metabolism and/or for protecting the body against external ingress, e.g. for the immune system.

The set may comprise an electronic detection unit. The cannula may be configured to be connected to or may be connectable to the electronic by wires or wireless.

A further aspect (third) relates to a method for determining the volume of a cavity in a living body. The method may comprise:
determining a volume and/or a pressure in a cavity of a living body and generating corresponding data using at least one cannula according to one of the embodiments mentioned above or a set according to one of the embodiments mentioned above,
preferably storing the generated data,
evaluating the generated data or the stored data, and
changing at least one parameter of an extracorporeal or an intra-corporeal blood pump which pumps blood through the cannula.

The flow of the pump (membrane, continuous (radial, axial, diagonal)) may be changed depending on the generated data, e.g. by changing operational parameters of the pump, e.g. voltage and/or current and/or periodicity of volume change of a membrane pump. Data may be transmitted from the electronic unit to a control unit of the pump via wires or wireless.

The method may further comprise:
determining volume and/or a pressure in at least two chambers, of the heart using two different portions of the cannula or using two of the cannulas, or
determining volume and/or pressure in at least three chambers of the heart using the cannula or using two of the cannulas, e.g. both cannulas may comprise the features of the cannula mentioned above (electrodes, etc.), or
determining volume and/or pressure in at least four chambers of the heart using two of the cannulas.

The physical parameters volume V and pressure p may be determined at least once for a series of beats of a heart. Determining may refer to non-SI units. Measure may refer to the usage of SI-units. Measuring may be a special case of determining.

Catheters with comparably small outer diameter may be used instead of comparably thick cannula(s) for blood transfer of for instance 1 l/min (liter per minute). The outer diameter of the catheter may be e.g. less than 10 French, less than 9 French, less than 8 French or less than 7 French. However, the outer diameter of the catheter may be more than 3 French.

For both variants, i.e. usage of cannulas for blood transport or usage of comparably thin catheters, it is possible to determine pressure and/or volume for at least two or for at least three or for all four chambers of the heart at least once or at least 10 times during one heart-beat. This may allow advanced diagnostic and/or treatment methods, e.g. it is possible to diagnose disease more exactly.

The method may be used for weaning a patient from a blood circuit support system. The unloading of the heart which is performed by the blood circuit support system may be reduced step by step. The blood circuit support system may also support the lung, e.g. using an oxygenator. PV loops may be determined to make decisions for changing parameters of the support system, for instance electrical parameters. PV loops may be considered for different degrees of unloading for making decisions about parameter changes. The unloading may be especially efficient if the patient is mobile and/or under activity. Corresponding arrangements of cannulas are provided below, e.g. arrangements which do not involve a cannula in the lower part of the body, e.g. in the legs. Usage of jugular veins may be preferred. Subclavian veins may provide an alternative.

The patient may carry the cannula and/or a power source and/or a pump and/or other unit(s) in his waistcoat or on a holder that comprises wheels. Thus, the patient can leave his bed and perform activity, e.g. walk on the isle, ride a stationary bicycle/home trainer ergometer, etc. This may accelerate recovery of the heart considerably.

A fourth aspect relates to a computer program product comprising instructions that are executable to cause a processor to perform the generation of data and/or the evaluation of data according to the method of any one of the embodiments mentioned above. The computer program product may be a transitory computer readable medium or a data stream or a signal stream that is transmitted via wire or wireless. The same technical effects that are valid for the method may also apply to the computer program product.

A fifth aspect relates to a computer system, comprising the computer program product, wherein the computer system may be configured to perform the generation of data and/or the evaluation according to the method of any one of the embodiments mentioned above. The same technical effects that are valid for the method may also apply to the computer system.

Alternatively, an electronic unit without processor may be used, e.g. using a unit which operates completely in analog technique or using a digital state machine without processor, preferably a finite state machine.

The proposed method and its embodiments may not be used for treatment of the human or animal body by surgery or therapy and/or may not be a diagnostic method practiced on the human or animal body. Alternatively, the proposed method and its embodiments may be used for treatment of the human or animal body by surgery or therapy and/or may be a diagnostic method practiced on the human or animal body.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference signs refer to the same technical features if not stated otherwise. As far as “may” is used in this application it means the possibility of doing so as well as the actual technical implementation. The present concepts of the present disclosure will be described with respect to preferred embodiments below in a more specific context namely heart and lung surgery. The disclosed concepts may also be applied, however, to other situations and/or arrangements in heart and lung surgery as well, especially to surgery of other organs.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale. In the drawings the following is shown in:

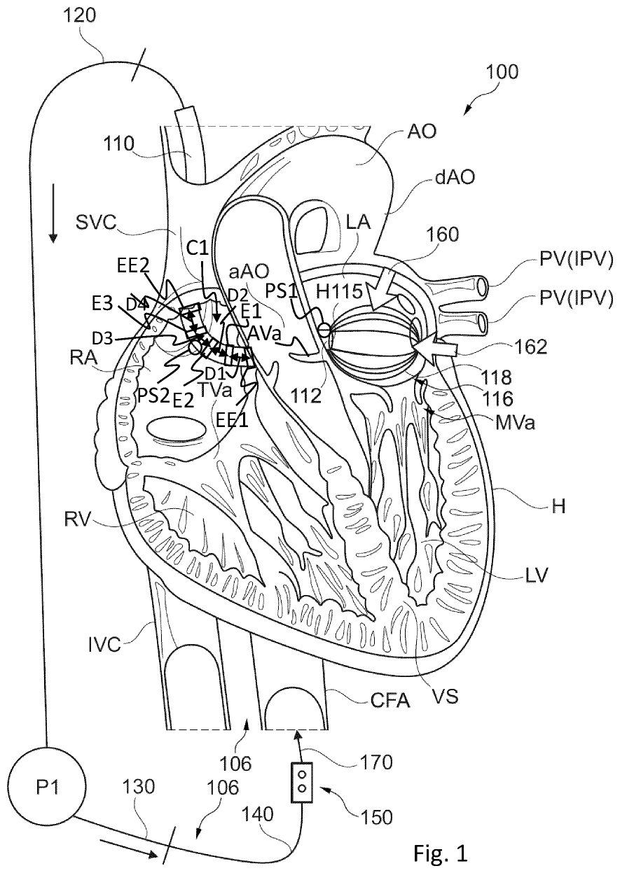

FIG. 1 an extra corporeal blood flow circuitry comprising two single lumen cannulas,

FIG. 2 an extra corporeal blood flow circuitry comprising a dual lumen cannula,

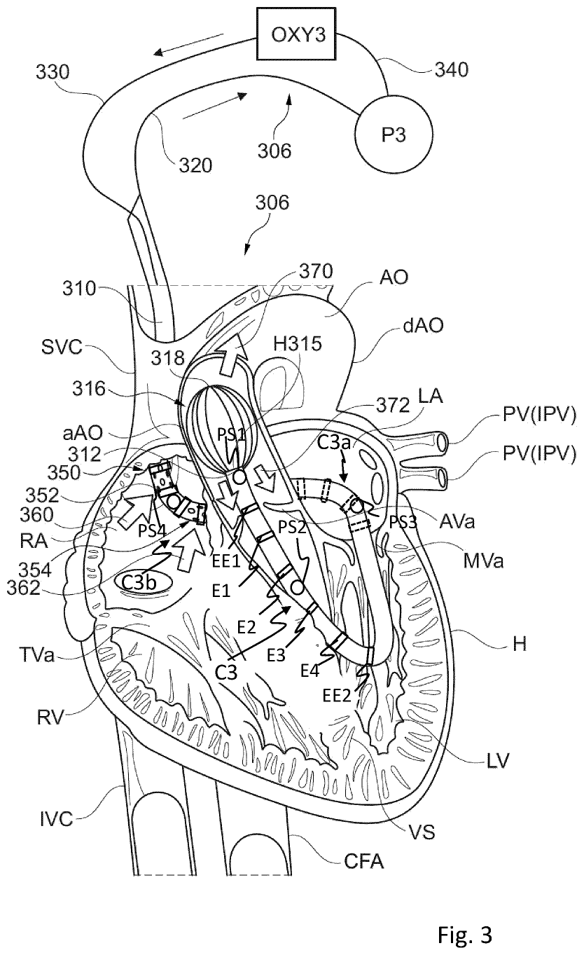

FIG. 3 an extra corporeal blood flow circuitry comprising a dual lumen cannula, a blood pump and an oxygenator,

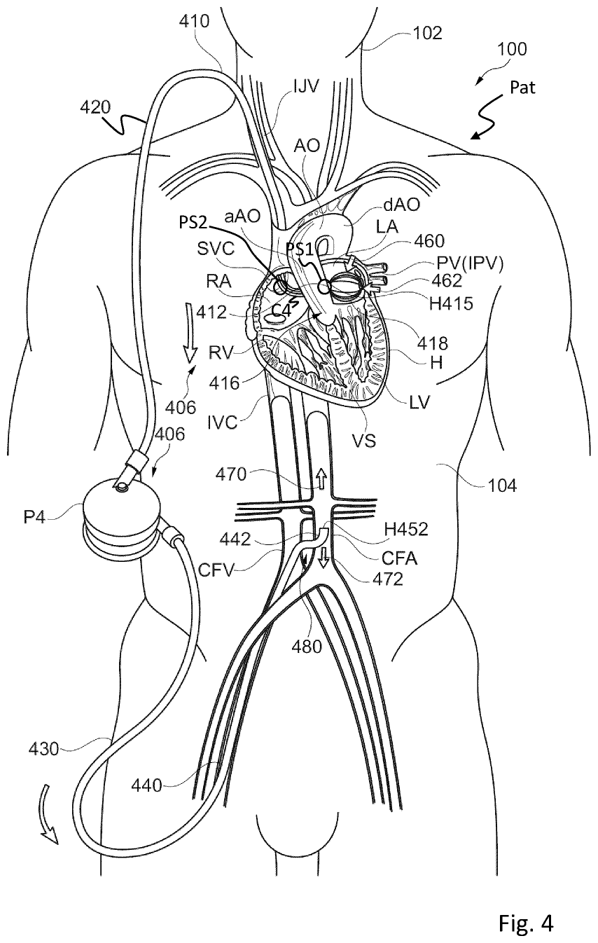

FIG. 4 a transcaval extra corporeal blood flow circuitry comprising two single lumen cannulas,

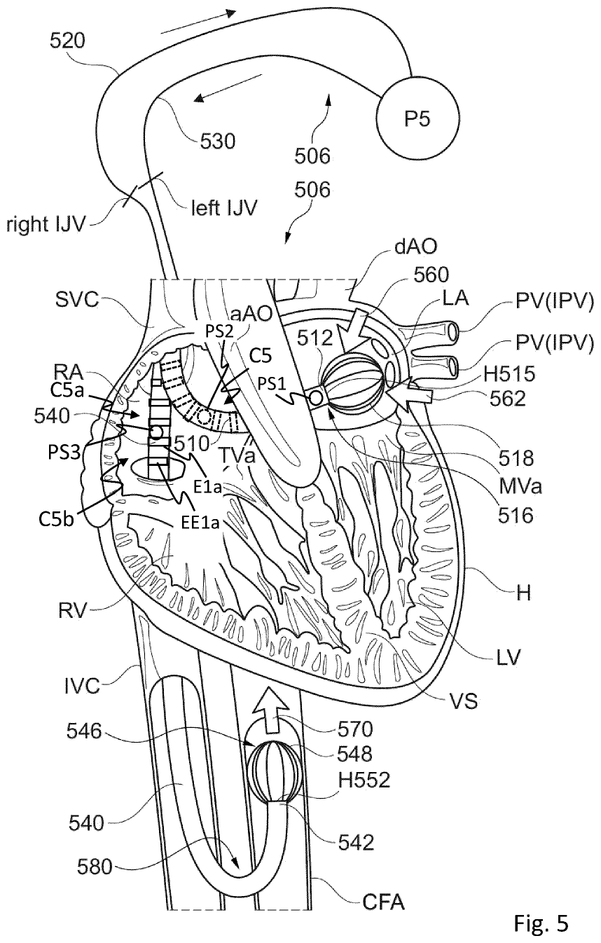

FIG. 5 a transcaval extra corporeal blood flow circuitry comprising two single lumen cannulas both inserted through jugular veins,

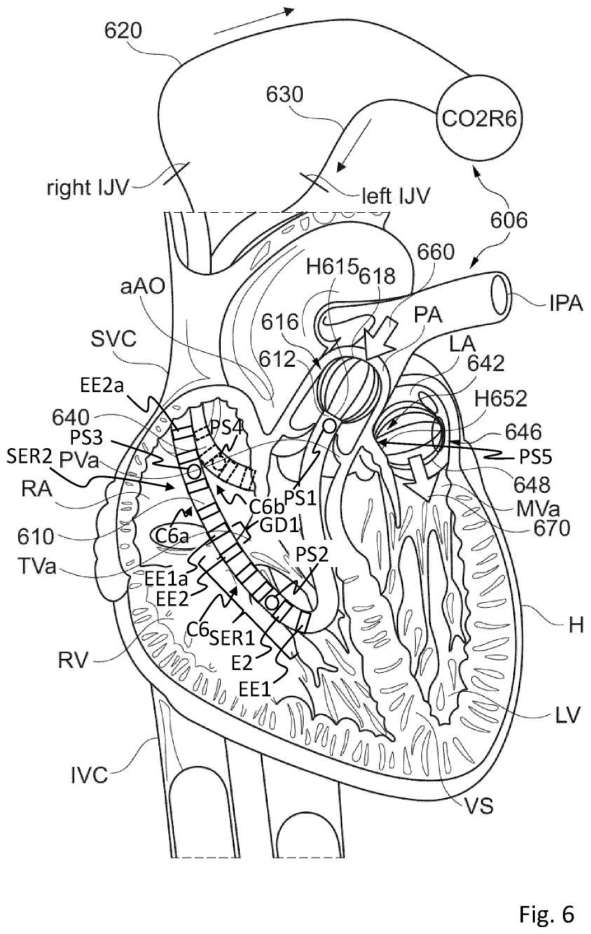

FIG. 6 an extra corporeal lung assist blood flow circuitry without a pump comprising two single lumen cannulas and a carbon dioxide removal device,

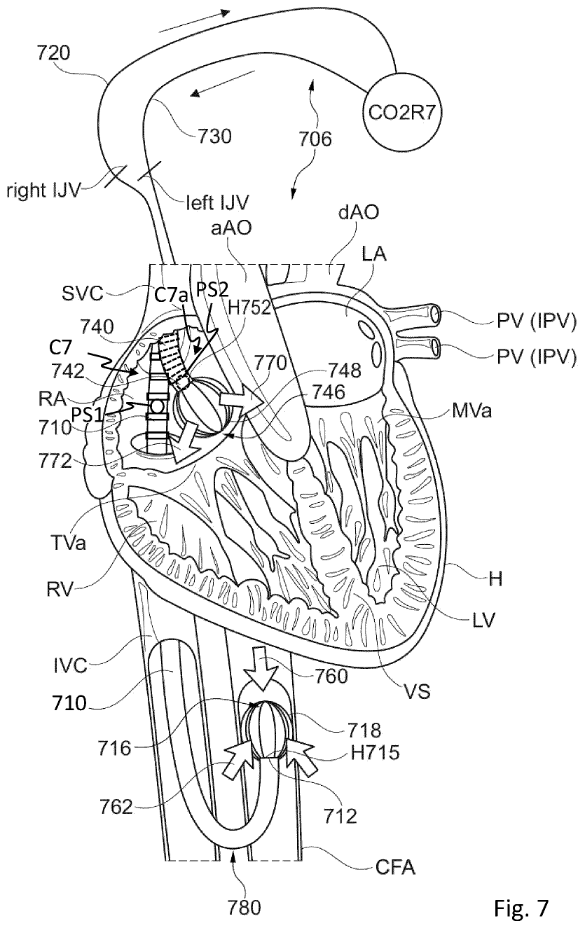

FIG. 7 a transcaval extra corporeal lung assist blood flow circuitry without a pump comprising two single lumen cannulas and a carbon dioxide removal device,

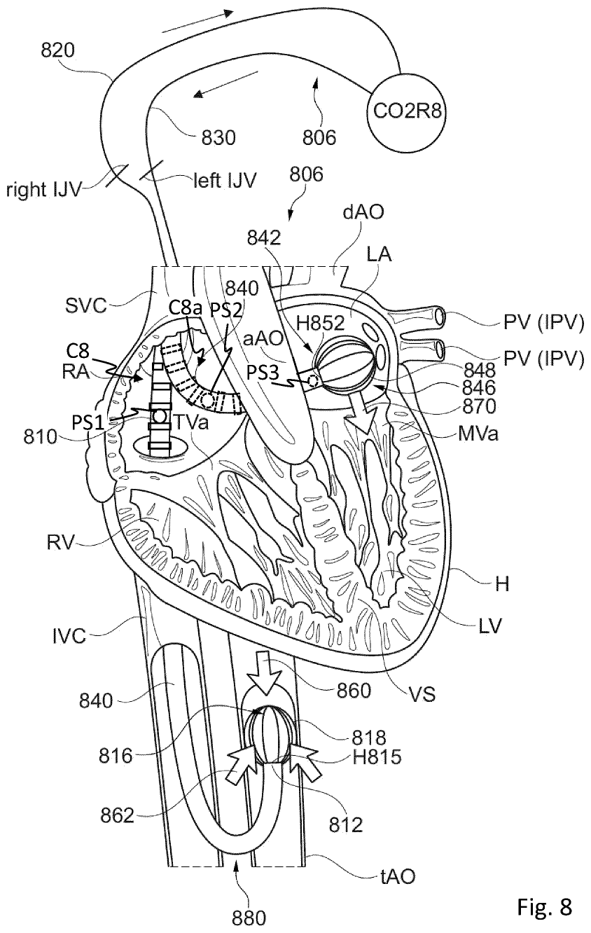

FIG. 8 a transcaval transseptal extra corporeal lung assist blood flow circuitry without a pump comprising two single lumen cannulas and a carbon dioxide removal device,

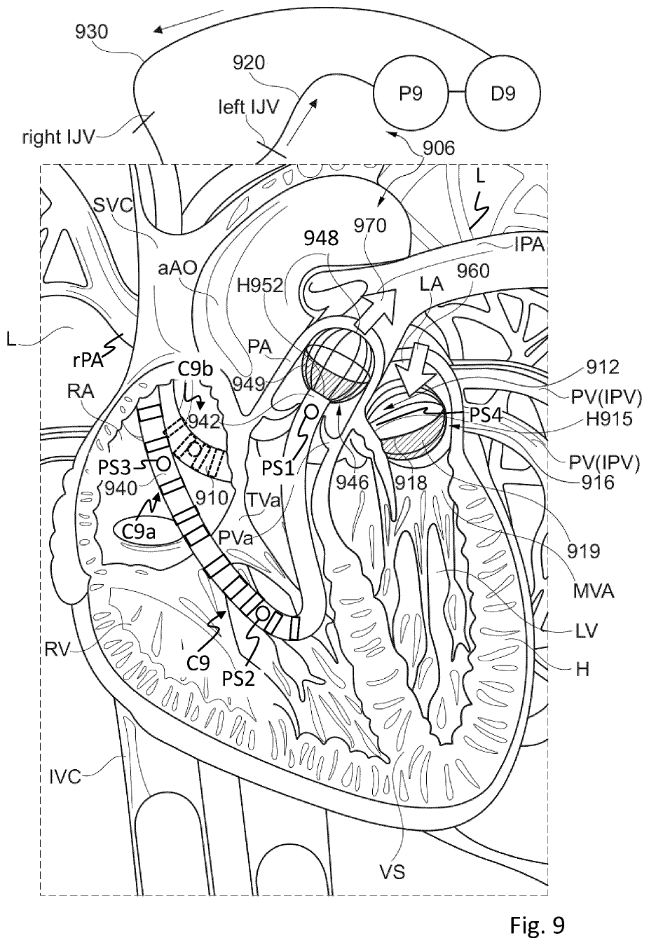

FIG. 9 an extra corporeal circular lung perfusion blood flow circuitry comprising two single lumen cannulas, a pump and a further device,

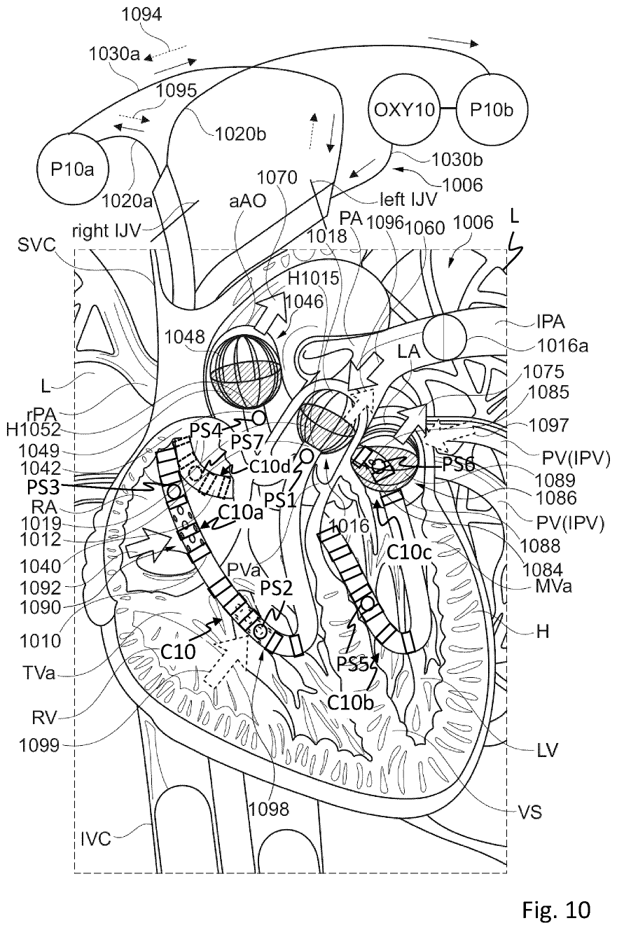

FIG. 10 an extra corporeal retrograde lung perfusion circular blood flow circuitry comprising two dual lumen cannulas, an alternative embodiment with antegrade lung perfusion, an alternative embodiment with lobe dedicated lung perfusion and an embodiment for right ventricle assist,

FIG. 11 a right ventricle assist circuitry with one inlet stage or with multi inlet stages,

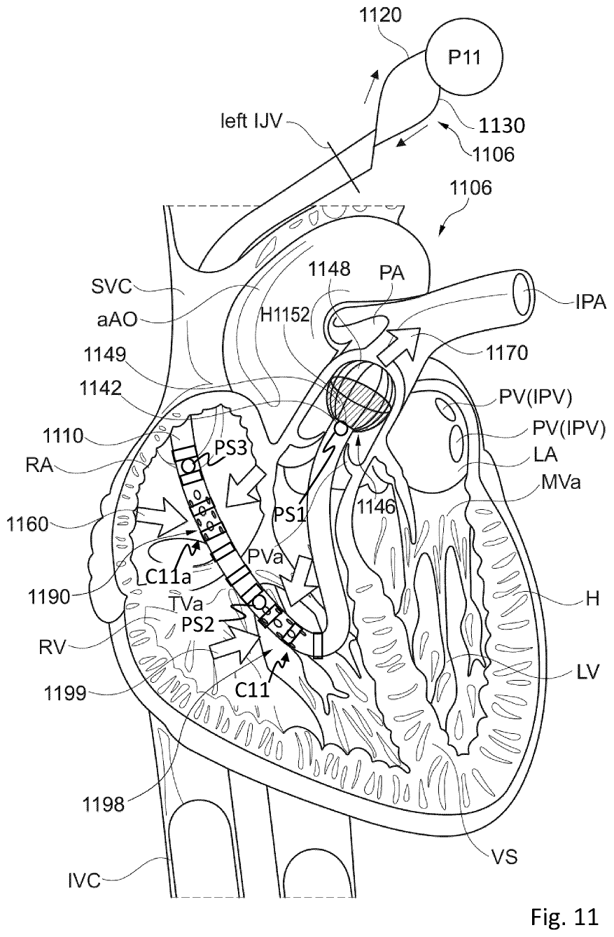

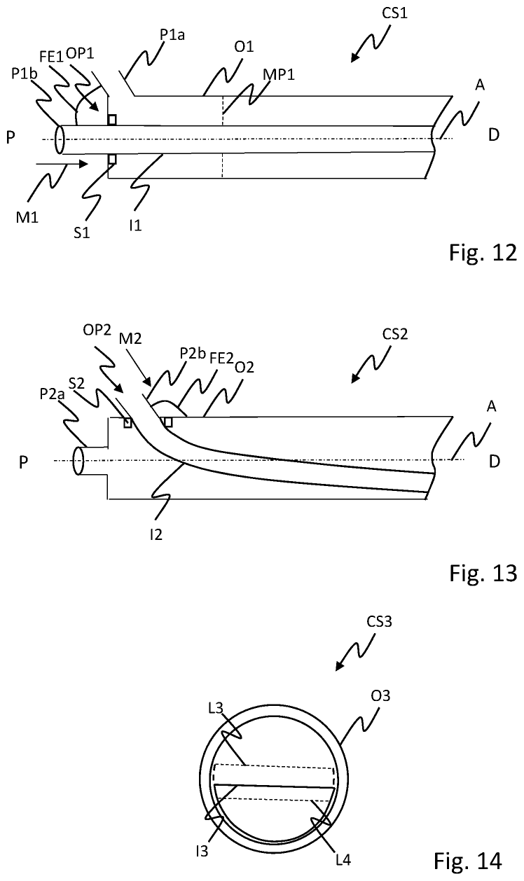

FIG. 12 a cannula system having cannulas that are arranged coaxially,

FIG. 13 a cannula system having an inner cannula that is arranged loosely within an outer cannula,

FIG. 14 a cross section of another cannula system,

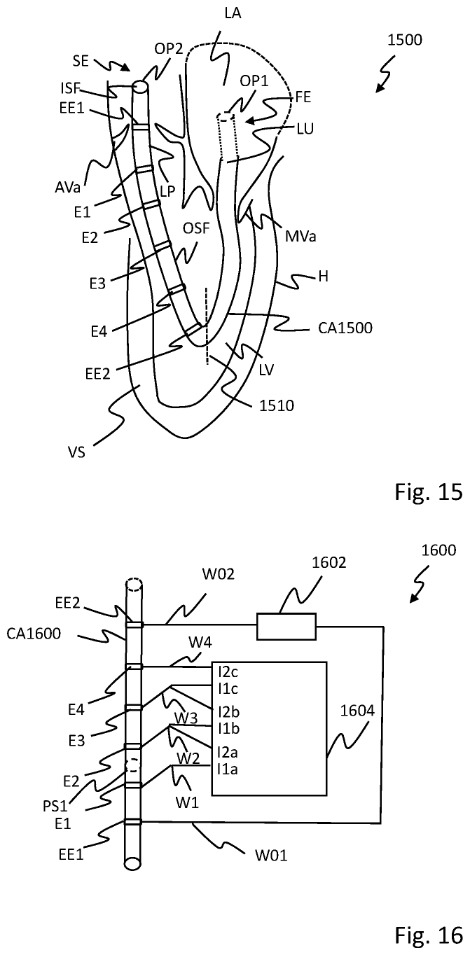

FIG. 15 a more general embodiment of using an endovascular catheter for support of the blood circuit and for detection of the volume of a cavity within the body of a living subject,

FIG. 16 an electronic circuitry allowing determination of the volume of a cavity, e.g. a cavity of the human heart,

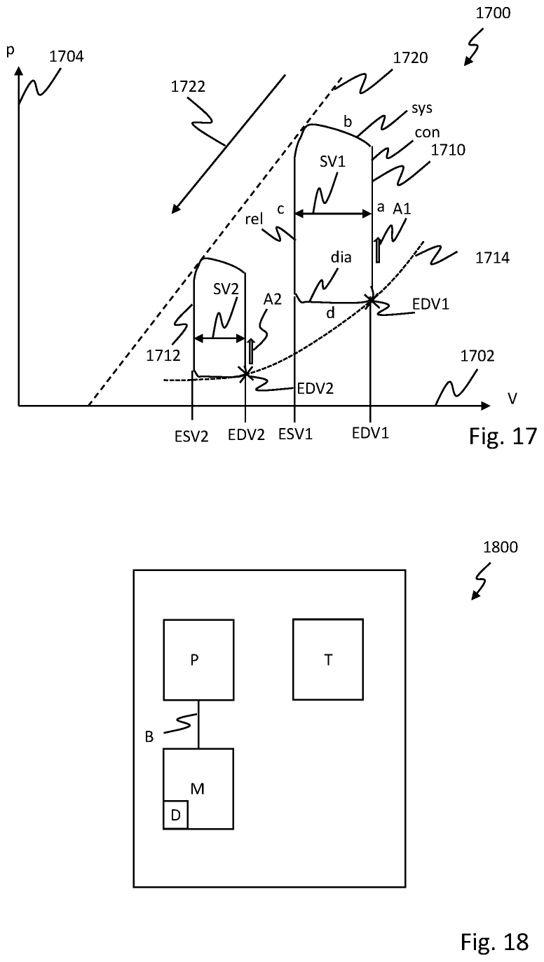

FIG. 17 an example of PV loops and for the adaption of a blood support circuit, and

FIG. 18 an example of a computer system which is used for determining or measuring the volume of the cavity in real time or in near real time.

### A) LEFT AND BI VENTRICLE ASSIST

FIG. 1 illustrates an extra corporeal circular blood flow circuitry 106 comprising a single lumen cannula 110 carrying a cage arrangement 116 near at least one inlet port, a blood pump P1 and a second single lumen cannula 140 that has at least one outlet port in an artery of the lower trunk. First, single lumen cannula 110 is inserted into a heart H through right internal jugular vein IJV, superior vena cava SVC, right atrium RA, transseptal, i.e. through the septum between right atrium RA and left atrium LA, into left atrium LA. A guide wire (not shown) may be used to guide cannula 110 to its final position. Alternatively, cannula 110 may be inserted through right subclavian vein. Blood is withdrawn by suction from left atrium LA through cannula 110, see arrows 160, 162.

Cannula 140 is inserted through the right femoral artery into common femoral artery CFA where blood is injected in a retrograde fashion into common femoral artery CFA, see arrows 170 and 172.

A body 100 comprises a head 102 and a trunk 104, see FIG. 4. Heart H of a patient Pat is located within the trunk 104 of patient Pat. Patient Pat may be a male or female adult or a child. Heart H comprises the following chambers: right atrium RA, right ventricle RV, left atrium LA and left ventricle LV.

The atrial septum is between right atrium RA and left atrium LA. The ventricle septum is between right ventricle RV and left ventricle LV.

The following valves of heart H are shown:
tricuspid valve TVa between right atrium RA and right ventricle RV,
mitral valve MVa between left atrium LA and left ventricle LV, and
aortic valve AVa between aorta AO and left ventricle LV.

A pulmonary valve PVa between right ventricle RV and pulmonary artery PA is omitted in order to not to obscure the view to the parts of heart H that are relevant in the shown embodiment. Left pulmonary veins PV, lPV are shown in FIG. 1. Blood that is enriched with oxygen comes from lung L into left atrium LA through pulmonary vein PV, lPV as well as through right pulmonary veins which are not shown. This is an exception in that a vein transports blood that comprises more oxygen than blood in a comparable artery. The description of heart H will not be repeated below. However, it is clear that this description is valid for all Figures, e.g. 1 to 11 and 15.

An optional inlet tip (not shown) may be mounted on distal end 112 of cannula 110. The inlet tip may comprise a plurality of inlet holes (also not shown) in its side wall. Additionally, there may be a hole within distal end 112 of the inlet tip. The sum of the cross-section areas of the holes of the inlet tip may be greater than the inner cross section area of cannula 110 at its distal end 112, for instance greater than twice the area or the triple of the area. This means that blood can be removed even if one or more of the inlet holes in the inlet tip is or are clogged. The inlet tip may be used to arrange electrodes within left atrium LA. The electrodes may be used to determine the volume V of the left atrium LA during beats of heart H. Using a pressure sensor PS1 at the end of cannula 110 may allow the detection of PV (pressure-volume) loops of left atrium LA for diagnostic and/or therapeutic purposes.

However, in other embodiments no separate inlet tip is used. Thus, there is only one inlet hole H115 at distal end 112 of cannula 110. This single inlet hole H115 may be surrounded by a cage arrangement 116.

Cage arrangement 116 is one possible example, e.g. a spherical cage or an elliptical cage in the expanded state of the cage arrangement 116. Other possible examples are cone shaped cages or cylindrical shaped cages. Cage arrangement 116 may comprise for instance between 6 to 12 flexible wires, beams or bars. There may be for instance 8 cage wires 118 that span a sphere. The sphere may prevent that the side wall of left atrium LA covers one of inlet holes of the inlet tip (not shown) or the inlet hole H115. Furthermore, cage arrangement 116 may fix distal end 112 of cannula 110 to the atrial septum AS. Thus, it is not possible that cannula 110 slides back into right atrium RA.

With reference still to FIG. 1, a tube 120 may be connected to a proximal end of cannula 110 and to an inlet of pump P1. Pump P1 may be a peristaltic pump or a centrifugal pump or another kind of pump, for instance a membrane pump or an axial pump. A tube 130 may be connected to an outlet of pump P1 and to the proximal end of cannula 140. Pump P1 may be an electrically driven pump. There may be a control unit that controls the pumping performance, for instance depending on an ECG (electrocardiography) signal or depending on another signal mentioned below, e.g. a signal indicating volume V and/or pressure p. Pump P1 may be operated in a pulsed or in a continuous mode.

Tubes 120, 130 may be made of a flexible material or of a more rigid material. Circuitry 106 may further include one or more blood filter units or units for dialysis of blood.

Cannula 140 may comprise an optional outlet tip (not shown) that has the same structure as inlet tip (not shown) of cannula 110. This means that the outlet tip may comprise a plurality of outlet holes (not shown) in its side wall and/or on its distal end. Alternatively, the outlet of cannula 140 may comprise only one central outlet hole (not shown) and/or a cage arrangement (not shown).

Extra care has to be taken because cannula 140 is inserted into an artery. Blood pressure is much higher in an artery compared to blood pressure in a vein. Furthermore, blood flow from a vein is continuously but blood flow in artery is pulsed. Pulsed mode of the pump may not be necessary because of the retrograde infusion.

However, pump P1 may be operated in a pulsed mode. Control may be performed depending on the rhythm of the heartbeat of heart H. A sensor may be used to detect the heartbeat, especially an electronic sensor, e.g. ECG, volume V and/or pressure p within heart H. If heart H is in a diastolic state the counter pressure against infusion of blood into artery CFA may be weak. Thus, this time point may be used preferred for infusion.

Retrograde infusion of blood may not be as advantageous as antegrade infusion because water divides of the lymphatic system are formed and because of the forming of turbulences. The formation of thrombus may be facilitated by retrograde infusion. The arrangement shown in FIG. 1 may be used for patients without lung problems. Furthermore, circuitry 106 supports the left part of heart H of the patient. The arrangement shown in FIG. 1 may be named pLVAD (percutaneous left ventricle assisted device). The advantages may outnumber the disadvantages of retrograde infusion.

Cannula 110 may comprise on its outer surface a plurality of electrodes of a conductance cannula portion C1:
a first source electrode EE1 which is arranged in the right atrium RA at a position which is adjacent to the atrial septum AS if cannula 110 is in its final position,
a first detection electrode E1 which is arranged with a predetermined distance D1 to source electrode EE1 but more proximally on cannula 110 compared to the position of the source electrode EE1,
a second electrode E2 which is arranged with a predetermined distance D2 to source electrode EE1 but more proximally on cannula 110 compared to the position of the electrode E1,
a third electrode E3 which is arranged with a predetermined distance D3 to electrode E2 but more proximally on cannula 110 compared to the position of the electrode E2,
optionally, one or more further electrodes,
a second source electrode EE2, which is arranged with a predetermined distance Dn (n is a natural number, e.g. in the range of 4 to 15) to the last electrode E(n−1) but more proximally on cannula 110 compared to the position of the last electrode E(n−1).

The distances D1 to Dn between adjacent electrodes EE1, E1, E2 etc. may be in the range of 4 mm (millimeter) to 12 mm. Distances D2 to D(n−1) may all have the same value. Distances D1 and D(n) may have the same value if compared to each other, for instance the same value as distances D2 to D(n−1). However, distances D1 and D(n) may be less or larger than one of the distances D2 to D(n−1).

Electrodes EE1, E1, E2, etc. may be used to determine the volume V of right atrium RA as is explained in more detail below, see FIGS. 15 to 18.

An optional pressure sensor PS2 may be used to determine or to measure the pressure p in right atrium RA. Thus, PV (pressure-volume) loops may be generated for right atrium RA. Pressure sensor PS2 may be arranged between two of the electrodes EE1, E1, E2 or at another appropriate position on cannula 110.

FIG. 2 illustrates an extra corporeal circular blood flow circuitry 206 comprising a dual lumen cannula system 210 and a blood pump P2. Dual lumen cannula system 210 may carry a cage arrangement 216 near at least one distal outlet port. First, an outer cannula of cannula system 210 may be inserted through right internal jugular vein rIJV, superior vena cava SVC, right atrium RA, transseptal, i.e. through the septum between right atrium RA and left atrium LA, into left atrium LA. Then, an inner cannula of cannula system 210 may be inserted through the outer cannula of cannula system 210 and then from left atrium LA through mitral valve MVa, left ventricle LV, through aortic valve AVa into ascending aorta aAO. A guide wire (not shown) may be used to guide the cannulas of cannula system 210 to its final position. Alternatively, outer cannula of cannula system 210 may be inserted through the right subclavian vein.

Blood may be withdrawn by suction from left atrium LA through an outer lumen of outer cannula of cannula system 210, see arrows 260, 262.

Blood may be pumped into ascending aorta aAO through an inner lumen of inner cannula of cannula system 210, see arrows 270, 272. Blood may be pumped in in a pulsed mode, preferably every time aortic valve AVa is closed. During the diastole, i.e. the heart refills with blood, there may be a first ejection of blood and during systole, i.e. contraction, there may be a normal or second ejection of blood out of the outlet holes of inner lumen of inner cannula of cannula system 210. Alternatively, blood may be only ejected during the systole or at another appropriate time. Alternatively a continuous blood flow may be generated, e.g. through hole H215.

Further to FIG. 2, a tube 220 may be connected to a proximal end of the outer lumen of cannula 210 and to an inlet of pump P2. Pump P2 may be a peristaltic pump or centrifugal pump or another kind of pump, for instance a membrane pump. A tube 230 may be connected to an outlet of pump P2 and to the proximal end of the inner lumen of cannula 210.

Pump P2 may be an electrically driven pump. The same features may be relevant for pump P2 which have been mentioned above for pump P1.

There may be a group of inlet holes 252 within the sidewall of the outer lumen within an intermediate portion of cannula 210, especially within an inlet portion 250 of outer cannula of cannula system 210. Inlet holes 252 may be arranged circumferentially on all sides of cannula 210. Inlet holes 252 may be arranged at a location of cannula 210 that is within left atrium LA if cannula 210 is arranged in place as shown in FIG. 2. Inner cannula of cannula system 210 may have a reduced outer diameter compared to the inner diameter of outer cannula of cannula system 210.

Cage arrangement 216 is one possible example, see description of cage arrangement 116 as given above. Thus, it is not possible that inner cannula of cannula system 210 slides back through aortic valve AVa into left ventricle LV. There may be only a single outlet hole H215 on distal end of cannula 210, e.g. within cage arrangement 216. Alternatively, a distal tip as mentioned above (FIG. 1) may be used on distal end of cannula 210, e.g. within cage arrangement 216.

Patient Pat may be able to walk because there are no cannulas in his/her legs or his groin. Furthermore, circuitry 206 supports the left part of heart H. The arrangement shown in FIG. 2 may be named pLVAD DL (percutaneous left ventricle assisted device dual lumen). The dual lumen cannula system 210 may be realized as shown. Alternatively, a dual lumen cannula may be used that is described in more detail below with regard to FIGS. 12 to 15.

An optional pressure sensor PS1 may be arranged at the distal end of inner cannula of cannula system 210. Pressure sensor PS1 may be used to determine the pressure within ascending aorta aAO.

A first conductive catheter portion C2 of cannula system 210 may comprise on the outer surface of the inner cannula a first series SER1 of electrodes EE1, E1, E2, E3, E4, optional further electrodes and an electrode EE2. The first series SER1 may be used to determine the volume V within the left ventricle LV of heart H. An optional pressure sensor PS2 may be arranged between two of these electrodes, e.g. between electrodes E2 and E3. With regard to the distances between electrodes EE1, E1, E2, E3, E4, optional further electrodes and electrode EE2 the same may valid as described above for the electrodes shown in FIG. 1.

A second conductive catheter portion C2a of cannula system 210 may comprise on the outer surface of the outer cannula a second series SER2 of electrodes EE1a, E1a, E2a, optional further electrodes and an electrode EE2a. The second series SER2 may be used to determine the volume V within the left atrium LA of heart H. An optional pressure sensor PS3 may be arranged between two of these electrodes, e.g. between electrodes E1a and E2a. With regard to the distances between electrodes EE1a, E1a, E2a, optional further electrodes and electrode EE2a the same may be valid as described above for the electrodes shown in FIG. 1.

A group distance GD1 between the last electrode EE2 of series SER1 and the first electrode EE1a of series SER2 may be larger than or may be larger than twice the largest distance between adjacent electrodes of the first series SER1.

A third conductive catheter portion C2b of cannula system 210 may comprise on the outer surface of the outer cannula a third series SER3 of electrodes EE1b, E1b, E2b, optional further electrodes and an electrode EE2b. The third series SER3 may be used to determine the volume V within the right atrium RA of heart H. An optional pressure sensor PS4 may be arranged between two of these electrodes, e.g. between electrodes E1b and E2b. With regard to the distances between electrodes EE1b, E1b, E2b, optional further electrodes and electrode EE2b the same may be valid as described above for the electrodes shown in FIG. 1.

A group distance GD between the last electrode EE2a of series SER2 and the first electrode EE1b of series SER3 may be less than the largest distance between adjacent electrodes of the second series SER2.

Thus, the volume V within three chambers LV, LA and RA may be determined, e.g. at the same time and/or within the same heart-beat period. Furthermore, if the pressure sensors PS2, PS3 and PS4 are used, it is possible to determine PV loops for these three chambers of the heart, for at least one of these chambers or for two of these chambers or cavities.

FIG. 3 illustrates an extra corporeal circular blood flow circuitry 306 comprising a dual lumen cannula 310 carrying a cage arrangement 316 near at least one outlet port, a blood pump P3 and an oxygenator OXY3. An outer cannula of cannula system 310 is inserted endovascularly through the right internal jugular vein rIJV, superior vena cava SVC, right atrium RA, transseptal, i.e. through the septum between right atrium RA and left atrium LA, into left atrium LA. Then, an inner cannula of cannula system 310 may be inserted through an outer lumen within the outer cannula and then further from left atrium LA through mitral valve MVa, left ventricle LV, through aortic valve AVa into ascending aorta aAO. A guide wire (not shown) may be used to guide cannulas of cannula system 310 to its final position. Alternatively, cannula system 310 may be inserted through the right subclavian vein.

Blood may be withdrawn by suction from right atrium RA through an outer lumen of cannula system 310, see arrows 360, 362. Blood may be pumped into the ascending aorta aAO through an inner lumen of cannula system 310, see arrows 370, 372. Blood may be pumped in in a pulsed mode, preferably every time aortic valve AVa is closed. During the diastole, i.e. heart H refills with blood, there may be a first ejection of blood and during systole, i.e. contraction, there may be a normal or second ejection of blood out of the outlet hole(s) of inner lumen of cannula 310. Alternatively, blood may be only ejected during the systole. Alternatively, a continuous blood flow may be generated, e.g. through a hole H315.

With reference further to FIG. 3, a tube 320 may be connected to a proximal end of the outer lumen of cannula system 210 and to an inlet of pump P3. Pump P3 may be a peristaltic pump or centrifugal pump or another kind of pump, for instance a membrane pump. A tube 340 may be connected to an outlet of pump P2 and to the inlet of an oxygenator device OXY3. Oxygenator device OXY3 may be used to enrich the oxygen content of the blood. Alternatively or additionally, oxygenator device OXY3 may also reduce carbon dioxide within blood. An outlet of oxygenator OXY3 may be connected to the proximal end of inner lumen of cannula system 310 by a tube 330.

Pump P3 may be an electrically driven pump. The same features which have been mentioned above for pump P1 may be valid for pump P3.

There may be a group of inlet holes 352 within the sidewall of the outer lumen of cannula system 310 within an intermediate portion of cannula 310, especially within an inlet portion 350 of cannula 310. Inlet holes 352 may be arranged circumferentially on all sides of outer cannula of cannula system 310. Inlet holes 352 may be arranged at a location of outer cannula of cannula system 310 that is within right atrium RA if outer cannula of cannula system 310 is arranged in place as shown in FIG. 3. An optional second inlet portion 354 may be arranged more distally than inlet portion 350. However, the inlet holes of second inlet portion 354 may also be arranged at a location of cannula system 310 that is within right atrium RA if cannula system 310 is arranged in place as shown in FIG. 3.

Cage arrangement 316 is one possible example. The same feature which are mentioned above for cage arrangement 116 may be valid for cage arrangement 316, e.g. fixing of distal end 312 within ascending aorta aAO. Furthermore, cage arrangement 316 mitigates “sandblasting” effects of the blood flowing out at distal end 312. There may be only a single outlet hole H315 on distal end of cannula 310, e.g. within cage arrangement 316. Alternatively, a distal tip as mentioned above (FIG. 1) may be used on distal end of cannula 310, e.g. within cage arrangement 316.

Patient Pat may be able to walk because there are no cannulas in his legs or his groin. Furthermore, circuitry 306 may support both the left part and the right part of heart H. The arrangement shown in FIG. 3 may be named as pBiVAD DL (percutaneous bi ventricle assist device dual lumen). Dual lumen cannula system 310 may be realized as shown. Alternatively, a dual lumen cannula may be used that is described in more detail below with regard to FIGS. 12 to 15.

With regard to conductive catheter portions C3, C3a and C3b the same may be valid as mentioned above for catheter portions C2, C2a and C2b.

FIG. 4 illustrates a transcaval extra corporeal circular blood flow circuitry 406 comprising a single lumen cannula 410 carrying a cage arrangement 416 near at least one inlet port H415, a blood pump P4 and a second single lumen cannula 440 that has at least one outlet port H452 in the artery of the lower trunk 104. First single lumen cannula 410 is inserted through the right internal jugular vein rIJV, superior vena cava SVC, right atrium RA, trans-septal, i.e. through the septum between right atrium RA and left atrium LA, into left atrium LA. A guide wire (not shown) may be used to guide cannula 410 to its final position. Alternatively, cannula 410 may be inserted through the right subclavian vein. Blood is withdrawn by suction from left atrium LA through cannula 410, see arrows 460, 462.

Cannula 440 may be inserted through the right femoral vein into the common femoral vein CFV and then transcaval via a transcaval passage 480 into common femoral artery CFA where blood is injected in a retrograde fashion into common femoral artery CFA, see arrows 470 and 472. Means may be used in order to support the vein and/or the artery openings that are part of transcaval passage 480. These means may be left within body 100 for further uses after removing cannula 440. An example for such means is a fixation set that is available within the market.

Body 100 comprises a head 102 and a trunk 104. Heart H of a patient Pat is located within trunk 104. Patient Pat may be a male or female adult or a child. The description of heart H is given above with regard to FIG. 1. This description is valid for all FIGS. 1 to 15.

An optional inlet tip (not shown) may be mounted on distal end 412 of cannula 410. The inlet tip was already mentioned above and reference is made thereto in order to avoid repetition. However, in other embodiments no separate inlet tip is used. Thus, there is only one inlet hole H415 at distal end 412 of cannula 410. This single inlet hole H415 may be surrounded by cage arrangement 416. The features of cage arrangement 416 may be the same as mentioned above for cage arrangement 116. The sphere of cage arrangement 416 may prevent that the side wall of left atrium LA covers inlet hole H415. Furthermore, cage arrangement 416 may fix distal end 412 of cannula 410 to the septum. Thus, it is not possible that cannula 410 slides back into right atrium RA.

With reference further to FIG. 4, a tube 420 is connected to a proximal end of cannula 410 and to an inlet of pump P4. Pump P4 may have the same features as pump P1. Thus, reference is made to the features mentioned above.

Tubes 420, 430 may be made of a flexible material or of a more rigid material. Circuitry 406 may further include one or more blood filter units or units for dialysis of blood. Cannula 440 may comprise an optional outlet tip (not shown) that has the same structure as inlet tip of cannula 410. This means that the outlet tip may comprise a plurality of outlet holes in its side wall and/or on its distal end. Additionally, cannula 440 may comprise a cage arrangement on its distal end 442. The cage arrangement may be realized as described above for instance for cage arrangement 116 or cage arrangement 546, see FIG. 5 and corresponding description. Alternatively, an outlet hole H452 may be used at the distal end of cannula 440 with or without a cage arrangement. The cage arrangement may fix cannula 440 at its final place. Furthermore, the cage arrangement may allow antegrade blood flow passing the distal end of cannula 440. Moreover, the cage arrangement around distal end 442 may mitigate “sandblasting” effects of the blood flowing out at distal end 442.

No extra care has to be taken because cannula 440 is inserted first into a vein in which there is comparably low blood pressure. However, the transcaval passage 480 has to be handled with care because blood pressure is much higher in an artery compared to blood pressure in a vein. Furthermore, blood flow in a vein is continuously but blood flow in an artery is pulsed. Pulsed mode of pump P4 is not necessary because of the retrograde blood infusion.

However, pump P4 may be operated in a pulsed mode. Control may be performed depending on the rhythm of the heartbeat of heart H. A sensor may be used to detect the heartbeat, especially an electronic sensor, e.g. ECG, volume V and/or pressure p sensor. If heart H is in a diastolic state the counter pressure against infusion of blood into artery CFA may be weak. Thus, this moment may be used for infusion.

Retrograde infusion of blood may not be as advantageous as antegrade infusion because of water divides of the lymphatic system that may be formed and because of the forming of turbulences. The formation of thrombus may be facilitated by retrograde infusion. Thus, appropriate care has to be taken in advance. The arrangement shown in FIG. 4 may be used for instance for patients Pat without lung problems. Other advantages may outnumber the disadvantages. Furthermore, circuitry 406 supports the left part of heart H of patient Pat. The arrangement shown in FIG. 4 may be named pLVAD transcaval (percutaneous left ventricle assisted device).

An optional pressure sensor PS1 may be arranged on the distal end of cannula 410 on a position which is arranged in left atrium LA when cannula 410 is in its final place. A conductive catheter portion C4 of cannula 410 may comprise on the outer surface of cannula 410 a series SER of electrodes EE1, E1, E2, optional further electrodes and an electrode EE2, details correspond to the electrodes which are shown in FIG. 5 within the right atrium RA on a corresponding conductive catheter C5. Electrodes EE1 and EE2 may be used as source electrodes. The same may be valid for Electrodes EE1 and EE2, EE1a, EE2a, etc. mentioned in the descriptions of other Figures. Electrodes E1 to En may be used as measurement electrodes as is described in more detail below, see description of FIG. 16. The series SER may be used to determine the volume V within the right atrium RA of heart H. An optional pressure sensor PS2 may be arranged between two of these electrodes, e.g. between electrodes E1 and E2. With regard to the distances between electrodes EE1, E1, E2, optional further electrodes and electrode EE2 the same may be valid as described above for the electrodes shown in FIG. 1.

FIG. 5 illustrates a transcaval extra corporeal circular blood flow circuitry 506 comprising a single lumen cannula 510 carrying a cage arrangement 516 near at least one inlet port, a blood pump P5 and a second single lumen cannula 540 that has at least one outlet port H552 in an artery of the lower trunk 104. First, single lumen cannula 510 may be inserted through right internal jugular vein IN, superior vena cava SVC, right atrium RA, transseptal, i.e. through the septum between right atrium RA and left atrium LA, into left atrium LA. A guide wire (not shown) may be used to guide cannula 510 to its final position. Alternatively, cannula 510 may be inserted through the right subclavian vein. Blood may be withdrawn by suction from left atrium LA through cannula 510, see arrows 560, 562.

Cannula 540 may be inserted through the left internal jugular vein IN, superior vena cava SVC, right atrium RA, inferior vena cava IVC and then transcaval via a transcaval passage 580 into common femoral artery CFA where blood is injected in a retrograde fashion into common femoral artery CFA, see arrow 570. Means may be used in order to support the vein and/or the artery openings that are part of transcaval passage 580. These means may be left within body 100 for further uses after removing cannula 540. An example for such means is a fixation set that is available within the market.

An optional inlet tip (not shown) may be mounted on distal end 512 of cannula 510. The inlet tip may comprise features of the inlet tip mentioned in the description of FIG. 1. However, in other embodiments no separate inlet tip is used. Thus, there is only one inlet hole H515 at the distal end 512 of cannula 510. This single inlet hole H515 may be surrounded by cage arrangement 516. Cage arrangement 516 may have the same features as mentioned above for cage arrangement 116. Furthermore, cage arrangement 516 may fix distal end 512 of cannula 510 to the atrial septum. Thus, it is not possible that cannula 510 slides back into right atrium RA.

Further to FIG. 5, a tube 520 is connected to a proximal end of cannula 510 and to an inlet of pump P5. Pump P5 may have the same features which are mentioned above for pump P1.

Tubes 520, 530 may be made of a flexible material or of a more rigid material. Circuitry 506 may further include one or more blood filter units or units for dialysis of blood.

Cannula 540 may comprise an optional outlet tip (not shown) that may have the same structure as the inlet tip of cannula 510. This means that the outlet tip may comprise a plurality of outlet holes in its side wall and/or on its distal end. Additionally, cannula 540 may have an optional cage arrangement 546 on its distal end 542. However, a single distal hole H552 may be used instead of an outlet tip. Hole H552 may be surrounded by optional cage arrangement 546.

Cage arrangement 546 is one possible example and may comprise features mentioned above for cage arrangement 116, e.g. cage wires 548. Furthermore, cage arrangement 546 may fix distal end 542 of cannula 540 within common femoral artery CFA and may allow antegrade blood flow of blood coming from heart H and/or from outlet hole H552. Furthermore, cage arrangement 546 may mitigate “sandblasting” effects of the blood flowing out at distal end 542 and may fulfill further functions mentioned already above.

Again, no extra care has to be taken because cannula 540 is inserted first into a vein in which there is comparably low blood pressure, see remarks mentioned above for cannula 440. Pump P5 may be operated in a continuous mode or in a pulsed mode, see remarks given above for pump P4.

Retrograde infusion of blood may not be as advantageous as antegrade infusion because of water divides of the lymphatic system and of the forming of turbulences. The formation of thrombus may be facilitated by retrograde infusion.

The arrangement shown in FIG. 5 may be used for patients Pat without lung problems. Furthermore, circuitry 506 supports the left part of the heart H of the patient. Mobility of patient Pat is possible because no cannulas in femoral veins or arteries are used. Compared with FIG. 4, the arrangement of FIG. 5 allows blood injection more central within body 100, e.g. into CFA, abdominal aorta AO or into thoracic aorta AO. The arrangement shown in FIG. 5 may be named pLVAD transcaval (percutaneous left ventricle assisted device). Retrograde blood flow may have the disadvantages mentioned above. Thus, corresponding measures have to be provided.

In other embodiments, it is possible to insert cannula 510 through the left internal jugular vein IN to the left atrium LA as described above and cannula 540 through the right internal jugular vein IN into the common femoral artery CFA.

An optional pressure sensor PS1 may be arranged on the distal end of cannula 510 on a position which is arranged in the left atrium when cannula 510 is in its final place. A conductive catheter portion C5 of cannula 510 may comprise on the outer surface of cannula 510 a series SER1 of electrodes EE1, E1, E2, optional further electrodes and an electrode EE2. Electrodes EE1 and EE2 may be used as source electrodes. Electrodes E1 to En may be used as measurement electrodes as is described below in more detail, see description of FIG. 16. Series SER1 of catheter portion C5 may be used to determine the volume V within the right atrium RA of heart H. An optional pressure sensor PS2 may arranged between two of these electrodes, e.g. between electrodes E1 and E2. With regard to the distances between electrodes EE1, E1, E2, optional further electrodes and electrode EE2 the same may be valid as described above for the electrodes shown in FIG. 1.

Alternatively or additionally, a conductive catheter portion C5a of cannula 540 may comprise on the outer surface of cannula 540 a series SER2 of electrodes EE1a, E1a, E2a, optional further electrodes and an electrode EE2a. Electrodes EE1a and EE2a may be used as source electrodes. Electrodes E1a to E(n)a may be used as measurement electrodes as is described below in more detail, see description of FIG. 16.

The series SER of catheter portion C5a may be used to determine the volume V within the right atrium RA of heart H. An optional pressure sensor PS3 may arranged between two of these electrodes, e.g. between electrodes E1a and E2a. With regard to the distances between electrodes EE1a, E1a, E2a, optional further electrodes and electrode EE2a the same may be valid as described above for the electrodes shown in FIG. 1.

Thus, conductance cannula portions C5 and C5a may be used simultaneously or as is appropriate in order to determine volume V of right atrium RA and/or pressure p of right atrium, e.g. in order to determine PV loops. Catheter portion C5 on cannula 510 may be used for instance if cannula 540 is not in place yet. Catheter portion C5a may deliver more exact results because cannula 540 is straight or almost straight within right atrium RA.

B) Lung Assist

FIG. 6 illustrates an extra corporeal circular blood flow circuitry 606 comprising two single lumen cannulas 610 and 620 and a carbon dioxide removal device CO2R6 but preferably no pump. Single lumen cannula 610 may carry a cage arrangement 616 near at least one inlet port H615 that is arranged within pulmonary artery PA. Second single lumen cannula 640 has at least one outlet port H652 within left atrium LA.

First, single lumen cannula 610 may be inserted through right internal jugular vein IJV, superior vena cava SVC, right atrium RA, right ventricle RV, through pulmonary valve PVa into pulmonary artery PA. A guide wire (not shown) may be used to guide cannula 610 to its final position. Alternatively, cannula 610 may be inserted through the right subclavian vein and then along the same way as described above. Blood may be withdrawn by suction from pulmonary artery PA through cannula 610, see arrow 660. A part of the blood that comes from right ventricle RV is pumped by heart H into pulmonary artery PA and is enriched in the lung with oxygen. Removal of carbon dioxide may be necessary for instance for patients Pat that have chronic obstructive pulmonary disease (COPD) or cystic fibrosis.

Cannula 640 may be inserted through left internal jugular vein IJV, superior vena cava SVC, right atrium RA, trans-septal, i.e. through the septum between right atrium RA and left atrium LA, into left atrium LA. A guide wire (not shown) may be used to guide cannula 640 to its final position. Alternatively, cannula 640 may be inserted through the right subclavian vein.

An optional inlet tip (not shown) may be mounted on distal end 612 of cannula 610. The inlet tip may have the same features as mentioned in the description of FIG. 1 for the corresponding inlet tip. However, in other embodiments no inlet tip is used. Thus, there is only one inlet hole H615 at the distal end 612 of cannula 610. This single inlet hole H615 may be surrounded by a cage arrangement 616. Cage arrangement 616 may have the same features as cage arrangement 116 mentioned above. The sphere of cage arrangement 616 may prevent that the side wall of left atrium LA covers inlet hole H615. Furthermore, cage arrangement 616 may fix distal end 612 of cannula 510 to pulmonary artery PA. Thus, it is not possible that cannula 510 slides back through pulmonary valve PV into right ventricle RV.

Further to FIG. 6, a tube 620 may be connected to a proximal end of cannula 610 and to an inlet of carbon dioxide removal device CO2R6 that removes carbon dioxide from the blood. A pump may not be necessary but may be used in another embodiment. A tube 630 may be connected to an outlet of carbon dioxide removal device CO2R6 and to the proximal end of cannula 640. Carbon dioxide removal device CO2R6 may contain a semipermeable membrane.

Tubes 620, 630 may be made of a flexible material or of a more rigid material. Circuitry 606 may further include one or more blood filter units or units for dialysis of blood. However, the natural blood pressure may not be sufficient to press the blood also through a filter device without using a pump.

Cannula 640 may comprise an optional outlet tip (not shown) that may have the same structure as the inlet tip (not shown) of cannula 610. This means that the outlet tip may comprise a plurality of outlet holes in its side wall and/or on its distal end. Additionally, cannula 640 may have an optional cage arrangement 646 on its distal end 642. A single outlet hole H652 may be used instead of outlet tip, e.g. with or without a cage arrangement 646. Blood with less carbon dioxide is injected into the left atrium LA through cannula 640 and mixes with oxygen rich blood that comes through the pulmonary veins PV from lung L.

Cage arrangement 646 is one possible example. Cage arrangement 646 may comprise features of cage arrangement 116, see e.g. cage wires 618. Furthermore, cage arrangement 646 may fix distal end 642 of cannula 640 within left atrium LA. Furthermore, cage arrangement 646 may mitigate “sandblasting” effects of the blood flowing out at distal end 64.

No extra care has to be taken because both cannulas 510 and 540 are inserted into veins in which there is comparably low blood pressure. Antegrade infusion is performed that has many advantages, i.e. no forming of water divides of the lymphatic system and less forming of turbulences. The formation of thrombus may be prevented by antegrade infusion. The arrangement shown in FIG. 6 may be used for patients Pat with lung problems. Mobility of patient Pat is possible because no cannulas in femoral veins or arteries are used. The arrangement shown in FIG. 6 may be named pECLA (percutaneous left extra corporeal lung assist). In other embodiments it is possible to insert cannula 610 through left internal jugular vein IJV/left subclavian vein to pulmonary artery PA as described above and cannula 640 through right internal jugular vein IJV/right subclavian vein to left atrium LA.

In another embodiment a pump is connected in series with carbon dioxide removal device CO2R6. This may allow to remove more carbon dioxide from the blood, for instance more than 30 percent compared to the content on the inlet of the carbon dioxide removal device CO2R6. This embodiment may be named ECCO₂R (extracorporeal CO₂ removal).

In a further embodiment an oxygenator device may be used instead of carbon dioxide removal device CO2R6 and preferably a pump is connected in series with the oxygenator. The oxygenator device may enrich the oxygen content in the blood and decreases the carbon dioxide content at the same time. This further embodiment may be named ECMO (extracorporeal membrane oxygenation).

An optional pressure sensor PS1 may be arranged on the distal end of cannula 510 on a position which is arranged in the pulmonary artery PA when cannula 610 is in its final place. A conductive catheter portion C5 of cannula 610 may comprise on the outer surface of cannula 610 a series SER1 of electrodes EE1, E1, E2, optional further electrodes and an electrode EE2. Electrodes EE1 and EE2 may be used as source electrodes. Electrodes E1 to En may be used as measure electrodes as is described below in more detail, see description of FIG. 16. The series SER1 of catheter portion C6 may be used to determine the volume V within the right ventricle RV of heart H. An optional pressure sensor PS2 may arranged between two of these electrodes, e.g. between electrodes E1 and E2. With regard to the distances between electrodes EE1, E1, E2, optional further electrodes and an electrode EE2 the same may be valid as described above for the electrodes shown in FIG. 1.

A conductive catheter portion C6a of cannula 610 may comprise on the outer surface of cannula 640 a series SER2 of electrodes EE1a, E1a, E2a, optional further electrodes and an electrode EE2a. Electrodes EE1a and EE2a may be used as source electrodes. Electrodes E1a to E(n)a may be used as measurement electrodes as is described below in more detail, see description of FIG. 16. The series SER2 of catheter portion C6a may be used to determine the volume V within the right atrium RA of heart H. An optional pressure sensor PS3 may be arranged between two of these electrodes, e.g. between electrodes E1a and E2a. With regard to the distances between electrodes EE1a, E1a, E2a, optional further electrodes and electrode EE2a the same may be valid as described above for the electrodes shown in FIG. 1.

A group distance GD2 between the last electrode EE2 of series SER1 and the first electrode EE1a of series SER2 may be comparably small, e.g. same or less than the largest distance between two adjacent electrodes in series SER1. Alternatively, the last electrode of series SER1 may be used as the first electrode of series SER2 resulting in a group distance of 0 mm (millimeter).

Alternatively or additionally, a conductive catheter portion C6b of cannula 640 may comprise on the outer surface of cannula 640 a series SER3 of electrodes EE1b, E1b, E2b, optional further electrodes and an electrode EE2b. Electrodes EE1b and EE2b may be used as source electrodes. Electrodes E1b to E(n)b may be used as measurement electrodes as is described below in more detail, see description of FIG. 16. The series SER3 of catheter portion C6b may be used to determine the volume V within right atrium RA of heart H. An optional pressure sensor PS4 may be arranged between two of these electrodes, e.g. between electrodes E1b and E2b. With regard to the distances between electrodes EE1b, E1b, E2b, optional further electrodes and electrode EE2b the same may be valid as described above for the electrodes shown in FIG. 1.

Thus, conductance cannula portions C6a and C6b may be used simultaneously or as is appropriate in order to determine volume V of right atrium RA and/or pressure p of right atrium RA, e.g. in order to determine PV loops. Catheter portion C6b may be used for instance if cannula 610 is not in place yet. Catheter portion C6a may be deliver more exact results because cannula 610 is straight or almost straight within right atrium RA. Furthermore, all three conductive catheter portions C6, C6a and C6b may be used at the same time or simultaneously in order to detect the respective volume V in right atrium RA or in right ventricle RV. Furthermore, an optional pressure sensor PS5 (not shown) may be used at distal end of cannula 640 in order to determine pressure within left atrium LA.

FIG. 7 illustrates a transcaval extra corporeal lung assist circular blood flow circuitry 706 comprising a single lumen cannula 710 carrying a cage arrangement 716 near at least one inlet port H715, a carbon dioxide removal device CO2R7 and a single lumen cannula 740 that has at least one outlet port H752 in the right atrium RA.

Cannula 710 may be inserted through right internal jugular vein IN, superior vena cava SVC, right atrium RA, inferior vena cava IVC and then transcaval via a transcaval passage 780 into common femoral artery CFA where blood with comparably high oxygen content is withdrawn from, see arrows 760, 762. A guide wire (not shown) may be used to guide cannula 710 to its final position. Alternatively, cannula 710 may be inserted through the right subclavian vein. Means may be used in order to support the vein and/or the artery openings that are part of transcaval passage 780. After removal of cannula 710, these means may be left within body 100 for further uses. An example for such means is a fixation set that is available within the market.

Single lumen cannula 740 may be inserted through left internal jugular vein IJV, superior vena cava SVC into right atrium RA. A guide wire (not shown) may be used to guide cannula 740 to its final position. Alternatively, cannula 740 may be inserted through right subclavian vein. Blood with reduced carbon dioxide content is ejected into the right atrium RA through cannula 740, see arrows 770, 772. This blood may then be pumped by heart H through right ventricle RV and pulmonary artery PA, see FIG. 6, to lung L of the patient Pat to whom body 100 belongs.

An optional inlet tip (not shown) may be mounted on distal end 712 of cannula 710. The inlet tip may comprise features mentioned in the description of FIG. 1 for the corresponding inlet tip. However, in other embodiments no inlet tip is used. Thus, there is only one inlet hole H715 at distal end 712 of cannula 710. This single inlet hole H715 may be surrounded by cage arrangement 716. Cage arrangement 716 may comprise features mentioned for cage arrangement 116. The sphere of cage arrangement 716 may prevent that the side wall of common femoral artery CFA covers inlet holes H715 of distal end 712. Furthermore, cage arrangement 716 may fix distal end 712 of cannula 710 to common femoral artery CFA. Thus it is not possible that cannula 710 slides back into transcaval passage 780. Alternatively, distal end 712 of cannula 710 may be arranged within thoracic part of aorta tAO or within abdominal part of aorta AO.

With reference further to FIG. 7, a tube 720 may be connected to a proximal end of cannula 710 and to an inlet of carbon dioxide removal device CO2R7. Carbon dioxide removal device CO2R7 may comprise a semipermeable membrane. A tube 730 may be connected to an outlet of carbon dioxide removal device CO2R7 and to the proximal end of cannula 740.

Tubes 720, 730 may be made of a flexible material or of a more rigid material. Circuitry 706 may further include one or more blood filter units or units for dialysis of blood. However, an additional pump may be necessary if a filter unit/dialysis unit is used.

Cannula 740 may comprise an optional outlet tip (not shown) that may have the same structure as inlet tip (not shown) of cannula 710. This means that the outlet tip may comprise a plurality of outlet holes 752 in its side wall and/or on its distal end. Additionally, cannula 740 may have an optional cage arrangement 746 on its distal end 742. Alternatively to an outlet tip, a single outlet hole H752 may be used with or without cage arrangement 746/cage wire(s) 748.

Cage arrangement 746 is one possible example. Cage arrangement 746 may comprise for instance features of cage arrangement 116 mentioned above. The sphere of cage arrangement 746 may prevent that the side wall of the right atrium RA is destroyed bay blood flowing out of hole H752. Furthermore, cage arrangement 746 may fix or attach distal end 742 of cannula 740 within right atrium RA to some degree.

No extra care has to be taken because both cannulas 710 and 740 are inserted first into a vein in which there is comparably low blood pressure. However, transcaval passage 780 has to be handled with care because blood pressure is much higher in an artery compared to blood pressure in a vein. Furthermore, blood flow from a vein is continuously but blood flow in an artery is pulsed. Antegrade infusion is performed that has many advantages, i.e. no forming of water divides of the lymphatic system may occur and less forming of turbulences may be present. The formation of thrombus may be prevented by antegrade infusion. The arrangement shown in FIG. 7 may be used for patients Pat with lung problems. Mobility of patient Pat is possible because no cannulas in femoral veins or arteries are used. The arrangement shown in FIG. 7 may be named pECLA (percutaneous left extra corporeal lung assist) transcaval. In other embodiments it is possible to insert cannula 710 through left internal jugular vein IJV/left subclavian vein to common femoral artery CFA as described above and cannula 740 through right internal jugular vein IJV into right atrium RA.

An optional pressure sensor may be arranged on the distal end of cannula 710 on a position which is arranged in common femoral vein CFA when cannula 710 is in its final place. A conductive catheter portion C7 of cannula 710 may comprise on the outer surface of cannula 710 a series SER1 of electrodes EE1, E1, E2, optional further electrodes and an electrode EE2. Electrodes EE1 and EE2 may be used as source electrodes. Electrodes E1 to En may be used as measurement electrodes as is described below in more detail, see description of FIG. 16. Series SER1 of catheter portion C7 may be used to determine the continuous changing volume V within right atrium RA of heart H. An optional pressure sensor PS1 may be arranged between two of these electrodes, e.g. between electrodes E1 and E2. With regard to the distances between electrodes EE1, E1, E2, optional further electrodes and electrode EE2 the same may be valid as described above for the electrodes shown in FIG. 1.

Alternatively or additionally, a conductive catheter portion C7a of cannula 740 may comprise on the outer surface of cannula 740 a series SER2 of electrodes EE1a, E1a, E2a, optional further electrodes and an electrode EE2a. Electrodes EE1a and EE2a may be used as source electrodes. Electrodes E1a to E(n)a may be used as measurement electrodes as is described below in more detail, see description of FIG. 16. The series SER2 of catheter portion C7a may be used to determine the volume V within right atrium RA of heart H. An optional pressure sensor PS3 may be arranged between two of these electrodes, e.g. between electrodes E1a and E2a. With regard to the distances between electrodes EE1a, E1a, E2a, optional further electrodes and electrode EE2a the same may be valid as described above for the electrodes shown in FIG. 1.

Thus, conductance cannula portions C7 and C7a may be used simultaneously or as is appropriate in order to determine volume V of right atrium RA and/or pressure p of right atrium, e.g. in order to determine PV loops. Catheter portion C7a may be used for instance if cannula 710 is not in place yet. Catheter portion C7 may be deliver more exact results because cannula 710 is straight or almost straight within right atrium RA. Moreover, an optional pressure sensor PS3 (not shown) may be used on distal end 712 of cannula 710 in order to determine the pressure within common femoral artery CFA or within thoracic part of aorta tAO or within abdominal part of aorta AO.

FIG. 8 illustrates a transcaval transseptal extra corporeal lung assist circular blood flow circuitry 806 comprising a single lumen cannula 810 carrying a cage arrangement 816 near at least one inlet port, a carbon dioxide removal device CO2R8 and a single lumen cannula 840 that has at least one outlet port in the left atrium LA.

Cannula 810 may be inserted through the right internal jugular vein IJ, superior vena cava SVC, right atrium RA, inferior vena cava IVC and then transcaval via a transcaval passage 880 into common femoral artery CFA, thoracic aorta tAO or abdominal aorta AO where blood with comparably high oxygen content is withdrawn from, see arrows 860, 862. A guide wire (not shown) and/or snares may be used to guide cannula 810 to its final position. Alternatively, cannula 810 may be inserted through the right subclavian vein. Means may be used in order to support the vein and/or the artery openings that are part of transcaval passage 880. These means may be left within body 100 after removing cannula 810 for further uses. An example for such means is a fixation set that is available within the market.

Single lumen cannula 840 may be inserted through the left internal jugular vein IJV, superior vena cava SVC, right atrium RA, trans-septal, i.e. through the septum between right atrium RA and left atrium LA, into left atrium LA. A guide wire (not shown) may be used to guide cannula 810 to its final position. Alternatively, cannula 810 may be inserted through the right subclavian vein. Blood with reduced carbon dioxide content may be ejected into left atrium LA through cannula 840, see arrow 870. This blood is then pumped by heart H through right ventricle RV and pulmonary artery PA, see FIG. 6, to lung L of the patient having body 100.

With regard to the following features, see the remarks for the corresponding features of FIG. 7:
optional inlet tip or inlet hole H815 (H718),
cage arrangement 816 (716) around inlet tip or inlet hole H815,
tube 820 (720), carbon dioxide removal device CO2R8 (CO2R7) and tube 830 (730),
optional outlet tip or outlet hole H852 (H752), and
cage arrangement 846 (746) around outlet hole H852 (H752).

Again, no extra care has to be taken because both cannulas 810 and 840 are inserted first into a vein in which there is comparably low blood pressure. However, transcaval passage 880 has to be handled with care because blood pressure is much higher in an artery compared to blood pressure in a vein. Furthermore, blood flow from a vein is continuously but blood flow in an artery is pulsed. Furthermore, antegrade infusion is performed that has many advantages. The arrangement shown in FIG. 8 may be used for patients Pat with lung problems. Mobility of patient Pat is possible because no cannulas in femoral veins or arteries are used. The arrangement shown in FIG. 8 may be named pECLA (percutaneous left extra corporeal lung assist) transcaval transseptal. In other embodiments it is possible to insert cannula 810 through left internal jugular vein IJV/left subclavian vein to common femoral artery CFA as described above and cannula 840 through right internal jugular vein IJV into right atrium RA.

In another embodiment a pump is connected in series with carbon dioxide removal device CO2R8. This allows to remove more carbon dioxide from the blood, for instance more than 30 percent compared to the content on the inlet of the carbon dioxide removal device CO2R8.

In a further embodiment an oxygenator is used instead of carbon dioxide removal device CO2R8 and preferably a pump is connected in series with the oxygenator device. The oxygenator device enriches the oxygen content in the blood and decreases the carbon dioxide content at the same time.

With regard to conductive catheter portions C8 and C8a the same may be valid as mentioned above for catheter portions C7 and C7a. Furthermore, an optional pressure sensor PS3 may be used on distal end 842 of cannula 840 in order to determine the pressure within left atrium LA. Moreover, an optional pressure sensor PS4 (not shown) may be used on distal end 812 of cannula 810 in order to determine the pressure within common femoral artery CFA or within thoracic part of aorta tAO or within abdominal part of aorta AO.

C) Lung Perfusion

An isolation of lung L is reached together with heart assist of heart H at the same moment for the circuitries that use percutaneous in-vivo lung perfusion (pIVLP). Thus, isolated perfusion and/or treatment of lung diseases is enabled, especially antegrade and/or retrograde, preferably also with switching between antegrade and retrograde or between retrograde and antegrade. However, if only a part of lung L is treated, the other part may function normal. There may be a lobe dedicated treatment or treatment of only a part of a lobe. This may allow to treat lung L without heart H assist/support and or without lung L support, e.g. without external blood oxygenation and/or without external carbon dioxide (CO₂) removal. Alternatively, partially or full heart H assist and/or lung L assist may be used even if only a part of lung L is treated, for instance.

FIG. 9 illustrates an extra corporeal lung perfusion circular blood flow circuitry 906 comprising two single lumen cannulas 910 and 940, a pump P9 and a further device D9. Single lumen cannula 910 may carry a cage arrangement 916 near at least one inlet port H915 that is arranged in left atrium LA. Second single lumen cannula 940 has at least one outlet port H952 within pulmonary artery PA. Circuitry 906 is a more theoretical embodiment because in addition a heart H assist would be necessary. However, there are many ways to realize such a heart H assist. One possibility is described below with reference to FIG. 10.

Cannula 910 may be inserted through the left internal jugular vein IJV, superior vena cava SVC, right atrium RA, trans-septal, i.e. through the atrial septum AS between right atrium RA and left atrium LA, into left atrium LA. A guide wire (not shown) may be used to guide cannula 910 to its final position. Alternatively, cannula 910 may be inserted through the right subclavian vein. Almost the whole blood that enters left atrium LA through the left and right pair of pulmonary veins PV may be taken in by cannula 910, see arrow 960, using a membrane 919 that is explained in more detail below.

The second single lumen cannula 940 may be inserted through the right internal jugular vein IN, superior vena cava SVC, right atrium RA, right ventricle RV, through pulmonary valve PVa into pulmonary artery PA. A guide wire (not shown) may be used to guide cannula 940 to its final position. Alternatively, cannula 940 may be inserted through the right subclavian vein and then along the same way as described above. Almost the whole blood that comes out of cannula 940 is injected into pulmonary artery PA, see arrow 970, using a membrane 949 that is explained in more detail below.

Device D9 may be an injection device that injects a medicament or a treatment substance, for instance for treating lung L cancer or other lung L diseases.

An optional inlet tip (not shown) may be mounted on distal end 912 of cannula 910. The inlet tip may comprise features mentioned for the corresponding inlet tip in the description of FIG. 1. However, in another embodiment no inlet tip is used. Thus, there is only one inlet hole H915 at distal end 912 of cannula 910. This single inlet hole H915 may be surrounded by cage arrangement 916. Using a cannula without a separate tip may allow high flow rates of a fluid that is drained into the cannula 910. Cage arrangement 916 is one possible example and may comprise the same features as cage arrangement 116. The sphere or other geometrical structure of cage arrangement 916 may prevent that the side wall of the left atrium LA covers one of inlet holes 915 of tip 914. Furthermore, cage arrangement 916 may fix distal end 912 of cannula 910 to the atrial septum AS. Thus, it is not possible that cannula 910 slides back through the atrial septum into right atrium RA.

Membrane 919 may cover only one half of cage arrangement 916, e.g. a half that is defined by two cage wires 918 that are arranged opposite to each other or nearly opposite. Examples of membranes that may be used are thin fabrics and/or thin plastic sheets.

Further to FIG. 9, a tube 920 may be connected to a proximal end of cannula 910 and to an inlet of pump P9. An outlet of pump P9 may be connected to an inlet of device D9. A tube 930 may be connected to an outlet of device D9 and to the proximal end of cannula 940. Device D9 may be used for instance for injecting a drug or medicament or treatment substance into lung L of patient Pat.

Tubes 920, 930 may be made of a flexible material or of a more rigid material. The circuitry 906 may further include one or more blood filter units or units for dialysis of blood.

Cannula 940 may comprise an optional outlet tip (not shown) that may have the same structure as inlet tip (not shown) of cannula 910. This means that the outlet tip may comprise a plurality of outlet holes in its side wall and/or on its distal end. Additionally, cannula 940 may have an optional cage arrangement 946 on its distal end 942.

However, in other embodiments no outlet tip is used. Thus, there is only one outlet hole H952 at distal end 942 of cannula 940. This single outlet hole H952 may be surrounded by cage arrangement 946. Cage arrangement 946 is one possible example and may have features of cage arrangement 116. The sphere of cage arrangement 946 and also the expelled blood prevent that the side wall of pulmonary artery PA covers one of outlet holes 952 of optional outlet tip 950. Furthermore, the sphere may prevent detrimental effects of outflowing blood to tissue of the pulmonary artery PA. Furthermore, cage arrangement 946 may fix distal end 942 of cannula 940 within pulmonary artery PA.

Membrane 949 may cover only one half of cage arrangement 946, e.g. a half that is between the distal end of cannula 940 and the mid of cage wires 948. Membrane 949 may comprise thin fabric or thin plastic sheets or other appropriate flexible material.

No extra care has to be taken because both cannulas 910 and 940 are inserted into veins in which there is comparably low blood pressure compared to the blood pressure in arteries. Antegrade infusion is performed into pulmonary artery PA that has many advantages because it corresponds to the natural direction of blood flow in lung L of the patient. The arrangement shown in FIG. 9 may be used for patients Pat with lung problems. Mobility of patient Pat is possible because no cannulas are used in femoral veins or arteries. The arrangement shown in FIG. 9 may be named pIVLP (percutaneous in vivo lung perfusion). In other embodiments it is possible to insert cannula 910 through right internal jugular vein IJV/right subclavian vein to left atrium LA as described above and cannula 940 through left internal jugular vein IJV/left subclavian vein to left atrium LA.

Alternatively, device D9 may be a CO₂ (carbon dioxide) removal device or an oxygenator. Furthermore, the pumping direction of pump P9 may be reversed, i.e. from antegrade to retrograde.

During treatment of lung L it is possible that patient Pat inhales a medicament or treatment substance in order to promote the treatment by the substance or medicament that flows through the vessels of the lung L and through the tissue of the alveoli. The fluid flow within circuitry 906 may comprise blood as a carrier substance. Alternatively, other carrier substances may be used, for instance based on saline and/or on water.

With regard to the arrangement of electrodes of conductance cannula portions C9, C9a and C9b the same features may apply as mentioned above for portions C6, C6a and C6c, e.g. determination of volume V and/or pressure p in right ventricle RV and/or in right atrium RA becomes possible during lung perfusion.

FIG. 10 illustrates an extra corporeal retrograde lung perfusion circular blood flow circuitry 1006 comprising two dual lumen cannulas 1010 and 1040, two pumps P10a, P10b and an oxygenator device OXY10. Dual lumen cannula 1010 carries a cage arrangement 1016 near at least one inlet port H1015 that is arranged within pulmonary artery PA and an inlet portion 1090 that is arranged in right atrium RA. Second dual lumen cannula 1040 has at least one outlet port H1052 within ascending aorta aAO and an outlet portion 1084 in left atrium LA. Circuitry 1006 allows for instance the removal of thrombus from lung L of patient. Alternatively, a chemotherapy of lung L may be performed, a stem cell treatment or cleaning of lung L.

Dual lumen cannula 1010 may be endovascularly inserted through right internal jugular vein IJV, superior vena cava SVC, right atrium RA, right ventricle RV, through pulmonary valve PVa into pulmonary artery PA. A guide wire (not shown) may be used to guide cannula 1010 to its final position. Alternatively, cannula 1010 may be inserted through the right subclavian vein and then along the same way as described above. Almost the whole blood that comes out of pulmonary artery PA may be extracted into an inner lumen of dual lumen cannula 1010, see arrow 1060, by using a membrane 1019 that is explained in more detail below. Other possibilities for insertion of a dual lumen cannula system 1010 will be explained below, e.g. first insertion of outer lumen and then insertion of inner lumen.

Dual lumen cannula 1040 may be endovascularly inserted through left internal jugular vein IJV, superior vena cava SVC, right atrium RA, trans-septal, i.e. through the septum between right atrium RA and left atrium LA, into left atrium LA. A guide wire (not shown) may be used to guide cannula 1040 to its final position. Alternatively, cannula 1040 may be inserted through the right subclavian vein. Almost the whole blood that exits the distal tip of the inner lumen of cannula 1040 may be injected into ascending aorta aAO, see arrow 1070, by using a membrane 1049 that is explained in more detail below. Other possibilities for insertion of a dual lumen cannula system 1040 will be explained below, e.g. first insertion of outer lumen and then insertion of inner lumen.

An optional inlet tip (not shown) may be mounted on distal end 1012 of cannula 1010. The inlet tip may comprise features as mentioned above in the description of FIG. 1. However, in another embodiment no inlet tip is used. Thus, there is only one inlet hole H1015 at distal end 1012 of cannula 1010, i.e. at the proximal end of the cage arrangement 1016. This single inlet hole H1015 may be surrounded by cage arrangement 1016. A single inlet hole H1015 may allow higher flow rates compared to the inlet tip that comprises lateral inlet holes.

Cage arrangement 1016 is one possible example. Other possible examples are described above with reference to cage arrangement 116. Cage arrangement 1016 may comprise for instance between 6 to 12 flexible wires, beams or bars. There may be for instance 8 cage wires 1018 that span a sphere. The sphere may prevent that the side wall of left atrium LA covers one or more of inlet holes of an inlet tip or the single end hole H1015 if no inlet tip is used. Furthermore, cage arrangement 1016 may fixe/attach distal end 1012 of cannula 1010 to pulmonary artery PA. Thus, it is not possible that cannula 1010 slides back through pulmonary valve PVa into right ventricle RV.

Membrane 1019 may cover only one half of cage arrangement 1016, e.g. a half that is between distal end 1012 of cannula 1010 and the mid of cage wires 1018 of cage arrangement 1016. Membrane 1019 may comprise a thin flexible fabric or a thin flexible plastic material.

Inlet portion 1090 may comprise a plurality of inlet holes that extend through the side wall of outer lumen of cannula 1010. Blood may be extracted by suction from right atrium RA into outer lumen of cannula 1010, preferably all blood or nearly all (for instance more than 90 percent of volume) blood that comes into right atrium RA, see arrow 1092. An optional cage arrangement may be arranged at inlet portion 1090.

Further to FIG. 10, a tube 1020a may be connected to a proximal end of inner lumen of cannula 1010 and to an inlet of pump P10a. An outlet of pump P10a may be connected to a proximal end of outer lumen of cannula 1040 using a tube 1030a.

A tube 1020b may be connected to a proximal end of outer lumen of cannula 1010 and to an inlet of pump P10b. An outlet of pump P10b may be connected to oxygenator device OXY10. A tube 1030b may be connected to an outlet of oxygenator device OXY10 and to the proximal end of inner lumen of cannula 1040. It is also possible to exchange the sequence of pump P10b and oxygenator device OXY10.

Pumps P10a, P10b may be peristaltic pumps, centrifugal pumps, membrane pumps or other kind of pumps. Oxygenator device OXY10 may enrich blood with oxygen wherein the blood comes out of right atrium RA and/or right ventricle RV (see inlet portion 1098 that is described in more detail below) and is then injected into ascending aorta aAO. Thus, the function of lung L may be fulfilled by oxygenator device OXY10 during treatment of lung L and the right heart H is supported.

Tubes 1020a, 1020b, 1030a, 1030b may be made of a flexible material or of a more rigid material. Circuitry 1006 may further include one or more blood filter units or units for dialysis of blood. Furthermore, a device may be used within circuitry 1006 for instance for injecting a drug or medicament and/or a treatment substance into lung L of patient Pat.

Cannula 1040 may comprise an optional outlet tip (not shown) that may have the same structure as inlet tip (not shown) of cannula 1010. This means that outlet tip may comprise a plurality of outlet holes in its side wall and/or on its distal end. Additionally, cannula 1040 may have an optional cage arrangement 1046 on its distal end 1042. If there is no outlet tip a single end-hole H1052 may be used at the distal end of cannula 1040, i.e. at the proximal end of optional cage arrangement 1046.

Cage arrangement 1046 is one possible example, see description of cage arrangement 116. The sphere of cage arrangement 1046 and also the expelled blood may prevent that the side wall of the ascending aorta aAO covers one of outlet holes of the optional outlet tip or outlet hole H1015. The sphere of cage arrangement 1046 may prevent that the injected blood damages the walls of the aorta AO, i.e. the “sand blasting effect” is prevented or mitigated even if no outlet tip is used. Furthermore, cage arrangement 1046 may fix distal end 1042 of cannula 1040 within ascending aorta aAO.

Membrane 1049 may cover only one half of cage arrangement 1046, e.g. a half that is between the distal end of cannula 1040 and the mid of cage wires 1048 of cage arrangement 1046. Thin fabric or thin plastic material may be used as material for membrane 1049.

A further outlet portion 1084 of cannula 1040 may comprises a plurality of outlet holes 1085 that extend through the sidewall of the outer lumen of cannula 1040. Outlet portion 1084 may be fluidically isolated from outlet hole H1052 within dual lumen cannula 1040 or within dual lumen cannula system 1040. Blood may be expelled through outlet portion 1084 into the two pairs of left and right pulmonary veins PV, see arrow 1075. Blood flow to left ventricle LV is thereby prevented by using membrane 1089. Outlet portion 1084 may be surrounded by an optional cage arrangement 1086. Cage arrangement 1086 is one possible example, see also description of cage arrangement 116. Cage arrangement 1086 may comprise for instance between 6 to 12 flexible wires, beams or bars. There may be for instance 8 cage wires 1088 that span a sphere. The sphere of cage arrangement 1086 and also the expelled blood may prevent that the side wall of left atrium LA covers one of outlet holes 1085. Moreover, the sand blasting effect may be prevented or mitigated cage arrangement 1086. Furthermore, cage arrangement 1086 may fix outlet portion 1086 of cannula 1040 within left atrium LA.

Membrane 1089 may cover only one half of cage arrangement 1086, e.g. a half that is defined by two cage wires 1088 that are arranged opposite to each other or nearly opposite. Membrane 1089 may comprise a thin flexible fabric or a thin flexible plastic material.

In summary, the following blood or other fluid flows may be established within circuitry 1006:
a) from pulmonary artery PA through inner lumen of cannula 1010 via pump P10a through outer lumen of cannula 1040 to left atrium LA, i.e. lung perfusion, and
b) from right atrium RA and/or right ventricle RV through outer lumen of cannula 1010 via pump P10b and OXY10 through inner lumen of cannula 1040 to ascending aorta aAO, e.g. external enrichment of blood with oxygen.

Flow a) is closed via right and left pulmonary veins PV, tissue of lungs L, right pulmonary artery rPA, left pulmonary artery lPA and pulmonary artery PA. Flow b) is closed via arteries of body 100, for instance common femoral artery CFA, tissues of body 100 and the veins of body 100, for instance common femoral vein CFV.

No extra care has to be taken because both cannulas 1010 and 1040 are inserted into veins in which there is comparably low blood pressure compared to blood pressure in arteries. The arrangement shown in FIG. 10 may be used for patients Pat with lung L problems. Mobility of patient Pat may be possible because no cannulas in femoral veins or arteries are used. The arrangement shown in FIG. 10 may be named pIVLP (percutaneous in vivo lung perfusion) retrograde. In other embodiments it is possible to insert cannula 1010 through internal jugular vein IJV/left subclavian vein to pulmonary artery PA as described above and cannula 1040 through right internal jugular vein IJV/right subclavian vein to ascending aorta aAO.

Conductance cannula portions C10, C10a, C10b, C10c, C10d may be used during retrograde lung perfusion in order to detect values of volume V within several cavities of heart H. Additionally or alternatively, corresponding pressures sensors may be used to determine pressure within the respective cavities and/or to generate respective PV (pressure-volume) loops. Details of conductance cannula portions C10, C10a, C10b, C10c, C10d are mentioned below for the antegrade lung perfusion mode but apply as well to the retrograde lung perfusion mode.

Furthermore, FIG. 10 illustrates an extra corporeal antegrade lung perfusion circular blood flow circuitry 1006 comprising two dual lumen cannulas 1010 and 1040, two pumps P10a, P10b and an oxygenator device OXY10. The proposed antegrade lung perfusion uses the arrangement 1006 of cannulas 1010 and 1040 as described above for retrograde lung perfusion. Reference is made to the description above in order to avoid unnecessary repetition. However, only the differences will be described. The main difference is that the direction of fluid flow in pump P10a is in the opposite direction now, see arrows 1094 and 1095. The direction of the blood flow or other fluid flow within the veins and arteries of the lung L is now antegrade.

Arrow 1096 shows that fluid is expelled from the distal end 1012 of cannula 1010 into pulmonary artery PA. Holes of a tip of cannula 1010 may be used as outlet holes for antegrade lung perfusion. Thus, optional tip of cannula 1010 may be an optional outlet tip for antegrade lung perfusion. However, alternatively, the single end-hole H1015 may be used as an outlet for antegrade lung perfusion. Membrane 1019 may direct the fluid flow into pulmonary artery PA completely or almost completely. Furthermore, membrane 1019 may have a valve function allowing blood/fluid flow from right ventricle into pulmonary artery but not in the inverse direction.

Arrow 1097 shows that blood or other fluid that comes out of pulmonary veins PV is extracted by suction into outer lumen of cannula 1040. Holes 1085 are inlet holes for antegrade lung perfusion and portion 1084 is an inlet portion for antegrade lung perfusion. Membrane 1089 may direct fluid flow from pulmonary veins PV completely or almost completely into outer lumen of cannula 1040. Thus, comparably toxic fluids for lung treatment may not flow into the blood circuitry.

In summary, the following flows may be established for antegrade lung perfusions within circuitry 1006:
a) from left atrium LA through outer lumen of cannula 1040 via pump P10a through inner lumen of cannula 1010 to pulmonary artery PA, i.e. lung L perfusion, and
b) from right atrium RA and/or right ventricle RV through outer lumen of cannula 1010 via pump P10b and OXY10 through inner lumen of cannula 1040 to ascending aorta aAO, e.g. external enrichment of blood with oxygen.

Flow a) is closed via pulmonary artery PA, right pulmonary artery rPA/left pulmonary artery lPA, tissue of lung L and right/left pulmonary veins PV. Flow b) is closed via arteries of body 100, for instance common femoral artery CFA, tissues of body 100 and the veins of body 100, for instance common femoral vein CFV.

Even for antegrade lung perfusion, no extra care has to be taken because both cannulas 1010 and 1040 are inserted into veins in which blood pressure is comparably low compared to blood pressure in arteries. The arrangement shown in FIG. 10 may be used for patients Pat with lung L problems. Mobility of patient Pat may be possible because no cannulas in femoral veins or arteries are used. The arrangement for antegrade lung perfusion shown in FIG. 10 may be named pIVLP (percutaneous in vivo lung perfusion) antegrade. Also for antegrade lung perfusion, it is possible to insert cannula 1010 through left internal jugular vein IJV/left subclavian vein to pulmonary artery PA as described above and cannula 1040 through right internal jugular vein IJV/right subclavian vein to ascending aorta aAO.

Furthermore, it is possible to switch fluid flow direction between antegrade and retrograde, starting with antegrade fluid flow in lung L vessels or with retrograde fluid flow whichever is appropriate. Switching may be repeated during one treatment session (for instance within one day) as often as necessary. Switching may ease the removal of at least one thrombus, especially of a blood thrombus.

For all embodiments mentioned with regard to FIG. 10 the following may be valid, i.e. retrograde lung perfusion, antegrade lung perfusion or dedicated lung perfusion. With regard to the arrangement of electrodes of conductance cannula portions C10, C10a and C10d the same features may apply as mentioned above for portions C6, C6a and C6c, e.g. determination of volume V and/or pressure p in right ventricle RV (using corresponding electrodes and/or using pressure sensor PS2) and/or in right atrium RA (using corresponding electrodes and/or using pressure sensor PS3 and/or PS7) becomes possible during lung perfusion or during other medical treatment. With regard to the arrangement of electrodes of conductance cannula portions C10b, C10c and C10d the same features may apply as mentioned above for portions C2, C2a and C2c, e.g. determination of volume V and/or pressure p in left ventricle LV (using corresponding electrodes and/or using pressure sensor PS5) and/or in left atrium LA (using corresponding electrodes and/or using pressure sensor PS6) and/or right atrium RA (using corresponding electrodes and/or pressure sensor PS7) becomes possible during lung perfusion or during other medical treatment.

For the first time, volume V and/or pressure p volume monitoring is possible within all four cavities of heart H, e.g. generating PV loops for all four cavities of heart H at the same time or for three or for two cavities of heart H. Patient P may be at rest thereby, for instance in a bed or at an operation table. However, mobility, e.g. walking and/or physical exercise, of patient P is also possible enabling monitored recovery of heart H/lung L of patient Pat and preventing degradation of muscles and/or of alveoli, etc. An optional pressure sensor PS1 may be used at the distal tip of cannula 1040 or on the distal tip of inner cannula of a dual lumen cannula system comprising an axially movable inner cannula within an outer cannula.

With regard to the electrodes of conductance cannula portion C10, C10a, C10b, C10c and C10d no modifications are necessary if compared with retrograde mode. Thus, measurement and/or detection of volume V and/or pressure p is possible during antegrade lung perfusion.

It is of course possible to use only some or only one of conductance cannula portion C10, C10a, C10b, C10c and C10d during lung perfusion. Thus, it is possible to omit one or more conductance cannula portion C10, C10a, C10b, C10c and C10d from cannula 1010 or 1040. The same is valid for retrograde lung perfusion mentioned above or for dedicated lung perfusion as mentioned in the following.

Moreover, FIG. 10 illustrates an extra corporeal lobe dedicated antegrade lung perfusion circular blood flow circuitry 1006 comprising two dual lumen cannulas 1010 and 1040, two pumps P10a, P10b and an oxygenator device OXY10. Dual lumen cannulas 1010 and 1040 may comprise axially fixed inner cannulas which are axially fixed to outer cannulas even during insertion of the dual lumen cannulas. Alternatively, dual lumen cannula systems 1010 and 1040 may be used which comprise axially movable inner cannulas in relation to outer cannulas. Thus, the outer cannula may be inserted first into body 100. Thereafter, inner cannula is inserted into outer cannula and further into body 100 in order to reduce overall trauma caused by insertion of dual lumen cannula system 1010, 1040 compared with a “fixed” dual lumen cannula 1010, 1040. The same is valid for all other dual lumen cannulas or dual lumen cannula systems mention in this application/patent.

The proposed lobe dedicated antegrade lung perfusion uses arrangement 1006 of cannulas 1010 and 1040 as described above for retrograde lung perfusion. Reference is made to the description above in order to avoid unnecessary repetition. However, only the differences will be described. One main difference is that the direction of blood/fluid flow in pump P10a is opposite to the direction mentioned above, see arrows 1094 and 1095. The other direction of fluid flow results in a change of the direction of the blood flow or other fluid flow within the veins and arteries of the lung.

A further difference is that cannula 1010 has a longer portion between inlet portion 1090 and distal end 1012 enabling an arrangement of a cage arrangement 1016a within left pulmonary artery lPA as shown in FIG. 10. Thus, cage arrangement 1016 may be omitted in dedicated mode. Cage arrangement 1016a is adapted to the diameter of left pulmonary artery lPA, i.e. it may be smaller in diameter than cage arrangement 1016. The other features of cage arrangement 1016a may be similar to the corresponding features of cage arrangement 1016 and would have an appropriate reduction in size, i.e. cage wires, membrane, optional outlet tip, outlet hole H1015, etc. However, it is also possible to use an inflatable balloon instead of cage arrangement 1016a.

The membrane of cage arrangement 1016a directs fluid flow that is expelled through inner lumen of cannula 1040 completely or almost completely into left pulmonary artery lPA. Furthermore, this membrane may have a valve function allowing blood flow from pulmonary artery PA also into left pulmonary artery lPA but not in the inverse direction. Right pulmonary arteries rPA are filled with blood coming from right ventricle RV into pulmonary artery PA.

Arrow 1097 shows that blood or other fluid that comes out of pulmonary veins PV is extracted by suction into outer lumen of cannula 1040 that is unchanged. Holes 1085 are inlet holes for dedicated antegrade lung perfusion and portion 1084 is an inlet portion for dedicated antegrade lung perfusion. Membrane 1089 directs fluid flow from pulmonary veins PV completely or almost completely into outer lumen of cannula 1040. Left pulmonary veins lPV will expel the fluid that is injected by cage arrangement 1016 and right pulmonary veins rPV will expel normal blood flow.

In summary, the following flows are established for dedicated antegrade lung perfusions within modified circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.
c) normal blood flow from right ventricle RV through pulmonary artery PA, through right pulmonary artery rPA, tissue of lung L back via right pulmonary vein rPV into left atrium LA.

Flow a) is closed via left pulmonary artery lPA, tissue of left lung lobe of lung L and left pulmonary veins lPV. Flow b) is closed via arteries of the body, for instance common femoral artery CFA, tissues of body 100 and the veins of body 100, for instance common femoral vein CFV.

Even for lobe dedicated antegrade lung perfusion, no extra care has to be taken because both cannulas 1010 and 1040 are inserted into veins in which there is comparably low blood pressure compared to blood pressure within arteries. The modified (dedicated) arrangement shown in FIG. 10 may be used for patients Pat with lung problems. Mobility of patient Pat may be possible because no cannulas in femoral veins or arteries are used. The arrangement for dedicated lobe antegrade lung perfusion shown in FIG. 10 may be named pIVLP (percutaneous in vivo lung perfusion) antegrade lobe dedicated. Also for dedicated lobe antegrade lung perfusion, it is possible to insert cannula 1010 through left internal jugular vein IJV/left subclavian vein to pulmonary artery PA as described above and cannula 1040 through right internal jugular vein IJV/right subclavian vein to ascending aorta aAO.

In another embodiment the right lobe of lung L may be flushed in the same way as described above for the left lobe of lung L. In this case, cage arrangement 1016a of cannula 1010 having a longer portion between inlet portion 1090 and distal end 1012 than shown in FIG. 10 would be arranged within right pulmonary artery rPA. Left pulmonary artery lPA would be filled with normal blood flow coming from right ventricle RV.

Furthermore, treatment of both lobes of lung L is possible sequentially, e.g. treating the left lobe first and then the right lobe or vice versa. Several changes of the lobes that are treated are possible as well. The afterload that arises within heart H may be reduced in this way. Further positive effects may be possible as well. Detrimental effects may be limited to only one lobe. After a period of recreation, the other lobe may be treated. Moreover, there may be diseases that require only the treatment of one lobe of lung L, for instance a thrombus in only one of the lobes of lung L.

For all three cases, i.e. retrograde lung perfusion, antegrade lung perfusion and dedicated lung perfusion, an optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during dedicated lobe antegrade lung perfusion.

For all three cases, i.e. retrograde lung perfusion, antegrade lung perfusion and dedicated lung perfusion, during dedicated treatment it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively, other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

A dedicated retrograde treatment of the lobes of lung L seems feasible if further measures are taken, for instance usage of at least one split tip cannula, for instance within the left pulmonary veins lPV or within the right pulmonary veins rPV, preferably comprising at least two border elements, preferably expandable border elements, which form a border between the natural blood circuit and the circuit for treatment liquid used for lung perfusion.

With regard to the electrodes of conductance cannula portion C10, C10a, C10b, C10c and C10d no modifications are necessary if compared with retrograde lung perfusion mode or with antegrade lung perfusion mode. Thus, measurement and/or detection of volume V and/or pressure p is possible during dedicated lung perfusion. PV (pressure-volume loops) may be generated as well.

D) Right Ventricle Assist

FIG. 11 illustrates a right ventricle assist circuitry 1106 with one inlet stage or with multi inlet stages. Circuitry 1106 comprises one dual lumen cannula 1110 and one pump P11. Dual lumen cannula 1110 may carry a cage arrangement 1146 near at least one outlet port H1152 that is arranged in pulmonary artery PA and at least one inlet portion 1190 that is arranged in right atrium RA. A second optional inlet portion 1198 may be arranged within right ventricle when cannula 1110 is arranged in place as shown in FIG. 11. It is also possible to use only inlet portion 1198 in right ventricle RV without having inlet portion 1190. Circuitry 1106 may be used for right ventricle assist.

Dual lumen cannula 1110 may be inserted through left internal jugular vein IN, superior vena cava SVC, right atrium RA, right ventricle RV, through pulmonary valve PVa into pulmonary artery PA. A guide wire (not shown) may be used to guide cannula 1110 to its final position. Alternatively, cannula 1110 may be inserted through the left subclavian vein and then along the same way as described above.

Almost the whole blood that comes out of inner lumen of cannula 1110 may be injected into pulmonary artery PA, see arrow 1170, by using a membrane 1149 that is explained in more detail below. Other possibilities for insertion of a dual lumen cannula system 1110 may be used, e.g. first insertion of outer lumen and then insertion of inner lumen.

Inlet portion 1190 may comprise a plurality of inlet holes that extend through the side wall of the outer lumen of cannula 1110. Blood may be extracted by suction from right atrium RA into outer lumen of cannula 1110, preferably all blood or nearly all (for instance more than 90 percent of volume) blood that comes into right atrium RA, see arrow 1160. Optional inlet portion 1198 may comprise a plurality of inlet holes that extend through the side wall of the outer lumen of cannula 1110. Blood may be extracted by suction from right ventricle RV into outer lumen of cannula 1110, preferably all blood or nearly all (for instance more than 90 percent of volume) blood that comes into left ventricle LV, see arrow 1199.

With reference further to FIG. 11, a tube 1120 may be connected to a proximal end of outer lumen of cannula 1110 and to an inlet of pump P11. An outlet of pump P11 may be connected to a proximal end of inner lumen of cannula 1110 using a tube 1130. Pump P11 may be peristaltic pump or a centrifugal pump.

Tubes 1120, 1130 may be made of a flexible material or of a more rigid material. The circuitry 1106 may further include one or more blood filter units or units for dialysis of blood.

Cannula 1110 may comprise an optional outlet tip (not shown). The outlet tip may comprise a plurality of outlet holes in its side wall and/or on its distal end. Additionally, cannula 1110 may have an optional cage arrangement 1146 on its distal end 1142. Alternatively a single outlet hole H1152 may be used at distal end 1142 with or without cage arrangement 1146.

Cage arrangement 1146 is one possible example, see remarks with regard to cage arrangement 116. The sphere of cage arrangement 1146 and also the expelled blood may prevent that the side wall of the pulmonary artery PA covers one of the outlet holes of optional outlet tip or single hole H1152 and/or that detrimental “sandblasting” effects occur. Furthermore, cage arrangement 1146 may fix distal end 1142 of cannula 1140 within pulmonary artery PA.

Membrane 1149 may cover only one half of cage arrangement 1146, e.g. a half that is between distal end 1142 of cannula 1140 and mid of cage wires 1148. Membrane 1149 may direct blood into pulmonary artery PA and may prevent that blood flows back into right ventricle RV. Membrane 1178 may have a valve function, e.g. if remaining blood comes from right ventricle RV it can pass between membrane 1149 and sidewalls of pulmonary artery PA. Membrane 1149 may comprise thin fabric or thin plastic material.

The following blood flow is established within circuitry 1106. Blood flows from right atrium RA and/or right ventricle RV through outer lumen of cannula 1110 via pump P11 back through inner lumen of cannula 1110 to pulmonary artery PA. This flow is closed via right and left pulmonary veins PV, arteries of body 100, for instance common femoral artery CFA, tissue of body 100 and veins of body 100, for instance common femoral vein CFV.

The arrangement shown in FIG. 11 may be used for patients Pat without lung problems. Mobility of patient Pat is possible because no cannulas in femoral veins or arteries are used. The arrangement shown in FIG. 11 may be named pRVAD (percutaneous right ventricle assist device) multi lumen.

It is possible to insert cannula 1110 through right internal jugular vein IJV/right subclavian vein to pulmonary artery PA as described above.

Conductance cannula portions C11 and C11a correspond to conductance cannula portions C6 and C6a as mentioned in the description of FIG. 6. Conductance cannula portions C11 and C11a may comprise the same electrode and/or pressure sensor configuration as conductance cannula portions C6 and C6a. Although inlet holes of inlet portions 1190 and/or 1198 are arranged between electrodes, measurement and/or detection of volume V and/or pressure p in right atrium RA and/or in right ventricle RV is possible.

Other applications of the proposed cage arrangements and/or dual lumen cannulas than these shown in FIGS. 1 to 11 are possible as well. All cannulas shown may be used with or without cages and/or with and without electrodes and/or with or without pressure sensors.

The cannula systems CS1 to CS3 that are described with reference to FIGS. 12 to 14 are further examples for dual lumen cannula systems shown in FIGS. 2, 3, 10 and 11. FIG. 12 illustrates a cannula system CS1 having an inner cannula I1 and an outer cannula O1 that are arranged coaxially relative to each other with inner cannula I1 arranged inside outer cannula O1.

Outer cannula O1 may be inserted into body 100 first, i.e. preferably before inner cannula I1 will be inserted. Only after the insertion of outer cannula O1 into body 100, preferably after the insertion of outer cannula O1 is completed, i.e. outer cannula O1 has reached its destination position, inner cannula I1 may be inserted into outer cannula O1 and then further beyond the distal end of outer cannula O1.

Both cannulas O1 and I1 are bendable up to a specific degree, i.e. they are bendable in radial directions. However, the diameter of cannulas O1 and I1 may not be variable in the sense that the area of the diameter cross section may be increased or decreased essentially.

Outer cannula O1 may have a circular or oval cross section along its entire length. A port P1a of outer cannula O1 may be arranged at a proximal P end of a sidewall of outer cannula O1. Proximal P end of outer cannula O1 may comprise a proximal surface, for instance a flat surface that may have an opening OP1. Opening OP1 may be arranged on the longitudinal axis A of outer cannula O1.

Inner cannula I1 may also have a circular or oval cross section along its entire length. A port P1b of inner cannula I1 may be arranged at a proximal P end of inner cannula I1. Inner cannula I1 may be inserted through opening OP1 into outer cannula O1, see movement direction M1. Thereby, inner cannula I1 may be arranged on the longitudinal axis A of outer cannula O1. At least one mounting portion MP1 or mounting elements may be arranged on an outer surface of inner cannula I1, e.g. protruding radially outward, and/or on an inner surface of outer cannula O1, e.g. protruding radially inward. Mounting portions MP1 may center inner cannula I1 within outer cannula O1.

A sealing element S1 may be used to seal cannula system CS1 proximally. Sealing element S1 may be arranged within opening OP1 or at another appropriate location. Sealing element S1 may be an O-ring in the simplest case. Alternatively, a multi-flap valve or a membrane may be used.

An optional fixation element FE1 may be arranged completely outside of outer cannula O1. Fixation element FE1 may have a first state in which axial movement M1 of inner cannula I1 relative to outer cannula O1 is possible or allowed and a second state that blocks such axial movement. Fixation element FE1 may operate automatically or semi-automatically or may be operated manually. Thus, fixation element FE1 may block axial movement if a predetermined length of inner cannula I1 is introduced into outer cannula O2. Alternatively, blocking may be performed manually at several positions of inner cannula I1 within outer cannula O1. It may be possible to bring fixation element FE1 back to the first state after it is in the blocking state.

Alternatively, fixation element FE1 may be arranged partly or completely within outer cannula O1. If it is completely within outer cannula O1 manual access to fixation element FE1 may be possible by operating elements. Alternatively, no manual access may be possible, i.e. fixation element FE1 may be operated in an automatic or semi-automatic mode depending for instance on the overlapping length of both cannulas I1 and O1.

Electrodes of at least one conductance cannula may be arranged on inner cannula I1 and/or on outer cannula O1. Furthermore, at least one pressure sensor may be arranged on inner cannula I1 and/or on outer cannula O1. Conductive wires may be embedded into the walls of inner cannula I1 and/or outer cannula O1 in order to connect electrodes/pressure sensors with further electronic units for the generation of measurement data.

FIG. 13 illustrates a cannula system CS2 having an inner (second) cannula I2 that is arranged loosely within an outer (first) cannula O2. Outer cannula O2 may be inserted into body 100 first, i.e. preferably before the inner cannula I2 will be inserted. Only after the insertion of outer cannula O2 into body 100, preferably after the insertion of outer cannula O2 is completed, i.e. outer cannula O2 has reached its destination position, inner cannula I2 is inserted into outer cannula O2 and then further beyond the distal end of outer cannula O2.

Both cannulas O2 and I2 are bendable up to a specific degree, i.e. they are bendable in radial directions. However, the diameter of cannulas O2 and I2 may not be variable in the sense that the area of the diameter cross section may be increased or decreased essentially.

Outer cannula O2 may have a circular or oval cross section along its entire length. A port P2a of outer cannula O2 may be arranged at a proximal P end of outer cannula O2 that may be arranged on the longitudinal axis A of outer cannula O2. The sidewall of outer cannula O2 may have an opening OP2 at its proximal P end. Opening OP2 may face laterally and or transversally relative to longitudinal axis A of outer cannula O2.

Inner cannula I2 may also have a circular or oval cross section along its entire length. A port P2b of inner cannula I2 may be arranged at a proximal P end of inner cannula I2. Inner cannula I2 may be inserted through opening OP2 into outer cannula O2, see movement direction M2. Thereby, the inner cannula I2 may be arranged loosely radially to longitudinal axis A of outer cannula O2. A mounting portion may not be necessary.

A sealing element S2 may be used to seal cannula system CS2 proximally. Sealing element S2 may be arranged within opening OP2 or at another appropriate location. Sealing element S2 may be an O-ring in the simplest case. Alternatively, a multi-flap valve or a membrane may be used.

An optional fixation element FE2 may be arranged completely outside of outer cannula O2. Fixation element FE2 may have a first state in which an axial movement M2 of inner cannula I2 relative to outer cannula O2 is possible or allowed and a second state that blocks such axial movement. Fixation element FE2 may operate automatically or semi-automatically or may be operated manually. Thus fixation element FE2 may block axial movement if a predetermined length of inner cannula I2 is introduced or inserted into outer cannula O2. Alternatively, blocking may be performed manually at several positions of inner cannula I2 within outer cannula O2. It may be possible to bring fixation element FE2 back to the first state after it is in the blocking state.

Alternatively, fixation element FE2 may be arranged partly or completely within outer cannula O2. If it is completely within outer cannula O2 manual access to fixation element FE2 may be possible by operating elements. Alternatively, no manual access may be possible, i.e. fixation element FE2 may be operated in an automatic or semi-automatic mode depending for instance on the overlapping length of both cannulas I2 and O2.

Electrodes of at least one conductance cannula may be arranged on inner cannula I2 and/or on outer cannula O2. Furthermore, at least one pressure sensor may be arranged on inner cannula I2 and/or on outer cannula O2. Conductive wires may be embedded into the walls of inner cannula I2 and/or outer cannula O2 in order to connect electrodes/pressure sensors with further electronic units for the generation of measurement data.

FIG. 14 illustrates a cross section of another cannula system CS3 that comprises an outer cannula O3 and an inner cannula I3. Outer cannula O3 has a circular inner cross section, preferably along its whole length. Alternatively, outer cannula O3 may have an oval or elliptic inner cross section, preferably along its whole length.

Inner cannula I3 has an outer cross section that is complementary to the inner cross section of outer cannula O3 and that leaves a lumen (first lumen in the claims) for the transport a fluid through outer cannula O3. If outer cannula O3 has an oval inner cross section, the outer cross section of inner cannula may be also oval or elliptic minus a part that is used for fluid transport in outer cannula O3.

The fluid may be blood or may comprise blood, for instance blood enhanced with a medicament or drug. Alternatively, other fluids than blood may be used.

Inner cannula I3 may have a flat outer surface that is arranged for instance along the longitudinal axis A of outer cannula O3. Alternatively, this flat surface of inner cannula I3 may be arranged on a side of the longitudinal axis A of outer cannula O3 on which the first lumen of the outer cannula O3 for fluid transport is located, see line L3. In a further alternative, the flat surface of inner cannula I3 may be arranged on a side of the longitudinal axis A of outer cannula O3 that is opposite to the side that comprises the main part of the first lumen of the outer cannula O3 for fluid transport, see line L4.

No mounting elements are necessary in cannula system CS3. However, it is possible to use mounting elements that position or fix the inner cannula I3 radially relative to outer cannula O3. Positioning would be easier than in cannula system CS1 because the complementary shapes of inner cannula I3 and outer cannula O3 may be used to enhance a specific positioning of inner cannula I3 within outer cannula O3.

Again, electrodes of at least one conductance cannula may be arranged on inner cannula I3 and/or on outer cannula O3. Furthermore, at least one pressure sensor may be arranged on inner cannula I3 and/or on outer cannula O3. Conductive wires may be embedded into the walls of inner cannula I3 and/or outer cannula O3 in order to connect electrodes/pressure sensors with further electronic units for the generation of measurement data D.

FIG. 15 illustrates a more general embodiment of using an endovascular cannula CA1500 for support of the blood circuit and for detection of the volume of a cavity within the body 100 of a living subject (e.g. a human or an animal). The cavity may be a chamber of heart H, for instance the left ventricle V.

Cannula CA1500 may comprise:
a lumen portion LP comprising and inner surface ISF and an outer surface OSF,
a first opening OP1 on a first end FE of the lumen portion LP, and
a second opening OP2 on a second end SE of the lumen portion LP.

In the example, second OP2 is an outlet of cannula (catheter) CA1500. However, in other embodiments, cannula CA1500 may be arranged within another cavity and/or introduced in a different manner, for instance via the aorta AO up to the apex of left ventricle LV of heart H.

The first opening OP1 and the second opening OP2 may have an inner diameter that allows endovascular blood circuit support of the blood circuit of a subject, e.g. patient Pat. A lumen LU may extend from the first end FE to the second end SE within the lumen portion LP. At least one series SER1 of electrodes EE1, E1 to E4 and EE2 may comprise at least 2, 3, 4, 5, 6, 7, 8, 9, 10, 11, 12, 13, 14 or at least 15 electrodes. FIG. 15 illustrates 6 electrodes wherein the end electrodes EE1 and EE2 are used for coupling in a source current and the electrodes E1 to E4 are used for measurement. Alternatively, “source” electrodes EE1 and EE2 may also be used for measurement purposes.

The series of electrodes EE1 to EE2 may be arranged along at least a part of an axial longitudinal axis of the lumen portion LP, preferably at the outer surface OSF. The at least one series SER1 of electrodes EE1 to EE2 may be configured to be used for the detection of the amount (value) of a volume V of a cavity within a body 100 of patient Pat. Examples for measurement methods are described below. However, other methods may also be used.

The length of cannula (catheter) CA1500 from the first end FE to the second end SE may be at least 50 centimeters or at least 70 centimeters at least 90 centimeters. The outer diameter of lumen portion LP or the diameter of lumen LU may be at least 10 French or at least 15 French or at least 20 French or at least 25 French or at least 30 French. Lumen LU may have a constant diameter over its length. Alternatively, the diameter of lumen LU may vary over lengths, for instance slightly increasing or slightly decreasing.

There may be at least one pressure sensor PS1 arranged within the series SER1 of electrodes EE1 to EE2 or at another appropriate place at cannula CA1500. The usage of at least one pressure sensor and of the electrodes EE1, E1 to E4 and EE2 allows to detect PV loops, see FIG. 17. PV loops may allow advanced diagnosis and/or treatment methods of patient Pat.

More than one series of electrodes may be used, see for instance FIG. 2 (comparably large group distance GD2) and FIG. 6 (comparably small group distance GD2).

There may be input holes or output holes between electrodes EE1, E1 to E4 and EE2, see for instance FIGS. 2, 3, 10 and 11. Thus, there may be a combination of electrodes and holes in one part of the cannula in order to enable measurement of volume V in combination with blood transport into cannula, e.g. CA1500, or out of cannula, e.g. CA1500.

According to FIG. 15, cannula CA1500 is a single lumen cannula. However, the same measurement principle of detecting or measuring volume V/pressure p may be used independent of the type of the endovascular cannula, for instance for dual lumen cannula, e.g. with an axially fixed or an axially movable inner cannula, see FIGS. 2, 3, 9, 10 and 11. A further cannula that may be used is a bidirectional cannula for bidirectional blood flow, e.g. changing the direction more than 20 times per minute, within a common part of the cannula which extends from a proximal portion to an intermediate portion of the bidirectional cannula.

Moreover, cannula CA1500 may comprise at least one variable diameter arrangement or volume expandable arrangement, for instance a cage or a balloon in order to attach the cannula, e.g. CA1500, at its final position within body 100. At least one membrane may be used which is attached to the cage, see FIGS. 9 to 11 as an example.

Cannula, e.g. CA1500, may be distributed separately from pumps, oxygenators and/or other medical devices. Alternatively, cannula CA1500 may be distributed within a set of medical devices in order to deliver components which are well adjusted to one another. The set may comprise the electronic unit which performs the detection and/or the measurement (SI units) of the volume V and/or of the pressure p and/or PV loops and/or of additional parameters within body 100 using the cannula, e.g. CA1500.

FIG. 16 illustrates an electronic circuitry 1600 allowing determination of the volume V of a cavity, e.g. a cavity of the human heart H. A cannula CA1600 may have the same parts as cannula CA1500, e.g. electrodes EE1, E1 to E4 and EE2, or more or less than six electrodes as well as an optional pressure sensor PS1. The arrangement of the pressure sensor PS1 between the electrodes may be chosen appropriately.

Electronic circuitry 1600 may be arranged outside of cannula CA1600, completely within cannula CA1600 or only partially within cannula CA1600. Only a pre-amplification of signals may be made within cannula CA1600.

Electronic circuitry 1600 may comprise: wiring W1 to W3, a signal source 1602 and a signal generating unit 1604.

Wiring W1 may form a conductive connection between electrode E1 and an input I1a of a first pre-amplifier. Wiring W2 may form a conductive connection between electrode E2 and a second input I2a of the first pre-amplifier and to a first input I1b of a second pre-amplifier. Wiring W3 may form a conductive connection between electrode E3 and a second input I2b of the second pre-amplifier and to a first input I1b of a third pre-amplifier. Wiring W4 may form a conductive connection between electrode E4 and a second input I2c of the third pre-amplifier. Two wires WO1 and W02 may be used as power supply wires.

Other wiring schemes may be used as well.

Only the electrodes, e.g. EE1, E1 to E(n), EE2, and the wiring, e.g. W1 to W3 and W01 and W02, may be arranged within cannula, e.g. CA 1600, for instance within a wall portion of cannula CA1600, or on the inner surface ISF and/or on the other surface OSF. The wires may be preferably shielded and/or distributed evenly in the circumference of the cannula, e.g. CA1600. Thus, cannula, e.g. CA1600 may be manufactured as a flexible cannula that is appropriate for the insertion methods described in this document.

Signal source 1602 may generate a varying current having constant amplitude. Signal source 1602 may comprise a trans-conductance amplifier, e.g. OTA (operational trans-conductance amplifier).

Signal generating unit 1604 may comprise further electrical units, e.g. band pass filters, wave filters, processors, e.g. see FIG. 18, and other components, e.g. power source unit.

The following measurement scheme may be used, see Baan et. al., “Continuous stroke volume and cardiac output from intra-ventricular dimensions obtained with impedance catheter”, Cardiovascular Research, 1981, 15, 328-334. The original equation for volume V by Baan, et. al. is:

V=1/alpha*rho*L\hat{ }2(G_meas−G_p) (0)

wherein:
rho is the blood resistivity,
L is the length between voltage sensing electrodes, E1, E2, E3 etc.,
alpha is a calibration factor,
G_meas is the conductance measured, and
G_p is the parallel conductance of muscle.

Gp may be measured by using a hypertonic saline bolus method or other methods. Calibration factor alpha may be determined by measuring stroke volume (SV), usually with a flow probe on the aorta AO or using another appropriate method. Thus, the Baan method is known since about 1980.

The volume V of the cavity may be segmented into several segments which form a series of segments. Formula or equation (1) may be applied for each segment. The volumes V_seg determined for each segment may then be summed up in order to calculate the overall volume V. The overall volume may be corrected for parts of the volume which are not detectable/measurable, for instance because of limited curvature of the catheter(s) or cannula(s) in the apex of cavities.

An improved method developed by Wei et al (see literature mentioned below) uses the complex conductance (i.e. admittance) to separate muscle from blood, since myocardium exhibits both resistance and capacitance, while blood is purely resistive. This improved method reduces errors due to position of the catheter in the ventricle. Wei et al., developed a non-linear equation for volume where gamma is a field form factor; this approach demonstrated improved accuracy even with radial misalignment of the catheter. This misalignment may be relevant for cannulas with conductance portions C1 to C11b. The modified equation for volume V is:

V=1/(1−(G_b/gamma))*rho*L\hat{ }2*(G_b) (1)

G_b=|Y|*sin(theta)−G_m (2)

G_m=C_m*(sigma_m)/(epsilon_m) (3)

Cm=|Y| sin(theta)/(2*Pi*f) (4)

The constants sigma_m and epsilon_m are the muscle conductivity and permittivity, respectively. |Y| is the absoluter value of the magnitude of the total measured conductance at an excitation frequency f and phase angle theta. Pi is 3.18

In VAD (Ventricle Assist Device) patients, echocardiography, which is routinely performed in VAD may be used to measure end-diastolic and end-systolic admittance in order to compute the field form factor gamma, as per Wei C et. al., “Volume catheter parallel conductance varies between end-systole and end-diastole”, IEEE Trans Biomed Eng 54: 1480-1489, 2007. 30, and/or Wei C, Valvano J W, Feldman M D, Pearce J A, “Nonlinear conductance volume relationship for murine conductance cannula measurement system”, IEEE Trans Biomed Eng 52: 1654-1661, 2005.

Further improved methods may be used as well, see for instance Dynamic correction for parallel conductance, G_P, and gain factor, alpha, in invasive murine left ventricular volume measurements. John E. Porterfield et. al., J. Appl Physiol 107: 1693-1703, 2009. Other measurement schemes may be used as well using electrodes arranged on a cannula or catheter, e.g. on an endovascular cannula or catheter.

Electrodes EE1, E1, E2, etc. may be made of thin layer of platinum, platinum black, platinum-iridium black or of other appropriate materials. In other embodiments, the two separate excitation electrodes EE1 and EE2 may be used also for measurement purposes. Polarization effects on these source or excitation electrodes EE1 and EE2 may be reduced for instance by using higher excitation frequencies.

A) Simple System—Only Volume Detection/Measurement

A simple system may perform only data recording, e.g. during patient Pat is mobile, i.e. he/she has left the operation-room where the cannula has or where the cannulas have been implanted. Recorded data D may be stored in a memory M, see FIG. 18, or may be transmitted to a recording station. Recorded data D may be used later for diagnostic purposes. Patient Pat may carry a waistcoat including electronic unit for data D recording. The waistcoat may also comprise a pump, an optional oxygenator and power source. Alternatively, a carriage may be used to allow mobility of patient Pat. Mobility may be extremely important for patient Pat in order to prevent degeneration of muscles, especially of heart muscles, or of lung L. Volume V may be measured in only one cavity or in several cavities, for instance of heart H.

B) Simple System—Volume Detection/Measurement and Pressure Detection/Measurement

See system A. In addition to system A, pressure is recorder as well, e.g. in order to allow the generation of PV loops, see FIG. 17 for diagnostic and/or therapeutic purposes. The pressure sensor may be a piezo resistant (preferred) or a piezoelectric or another appropriate sensor. At least one pressure sensor or more than one pressure sensor may be used. According to a variant, pressure sensors are used on the cannula(s) but no electrodes of conductance cannula portions.

C) Advanced System

See systems A or B plus data D transmission, e.g. via WLAN (Wireless Local Area Network), ZigBee, Bluetooth) or other types of electronic data transmission.

D) High End System

See systems mentioned in A, B or C and automatic control system for e.g. weaning/unloading. There may be a closed control loop between recorded data D and parameter control of the blood circuit support system 106 to 1106.

Shift keying methods may be used for data D transmission via wires or wireless, e.g. ASK (amplitudes shift keying), FSK (frequency shift keying) etc. Output trans-conductance amplifiers (OTA) may be used. Input differential amplifiers (instrumentation amplifier, amps) may be used. Band-pass filters and/or wave filters may be used. Class E amplifiers may be used. Internal serializers may be used, e.g. I²L.

Adapted products of the company CD Leycom may be used, for instance software Conduct NT or higher release. CD Leycom offers a wide range of catheters with different electrode spacing to get the maximum results (see table). These catheters may be adapted to the cannulas mentioned in this application, e.g. the pigtail has to be removed, so that the first electrode on the cannulas is as close as possible to the apex of the ventricle for instance.

Optimally, the effective measuring catheter/cannula length C1a to C11b may match the ventricular long axis (as measured e.g. by echocardiography):
Minimum: 4*L+2*La [+10.0 (offset because of limited curvature of catheter, width of pigtail of catheter)] (mm)
Maximum: 7*L+2*La [+10.0 (offset)] S(mm)
L=Electrode spacing, and
La=Spacing (e.g. 3 mm) between the driving electrodes EE1a, EE1b.

CD Leycom offers catheters with electrode spacing ranging from 6 to 12 mm. Other values may be used as well. These spacings may also be used for a cannula.

The proximal driving electrodes may be positioned above the aortic valve AVa:

| 4 French type | Electrode spacing | Long Axis | |
|---|---|---|---|
| without lumen | in mm | in mm | Application |
| CA-41063-PN* | 6 | 46 to 64 | Children |
| CA-41103-PN | 10 | 56 to 86 | Normal adults |
| 7 French type | | | |
| without lumen | | | |
| CA-71083-PN | 8 | 48 to 72 | Small adults |
| CA-71103-PN | 10 | 56 to 86 | Normal adults |
| 7 French type with | | | |
| lumen | | | |
| CA-71083-PL | 8 | 48 to 72 | Small adults |
| CA-71103-PL | 10 | 56 to 86 | Normal adults |
| CA-71123-PL | 12 | 64 to 100 | Large adults |

*Segment 3 of this catheter measures 12 mm to fit in the pressure-sensor

The diameters given in the table for catheters have to be adjusted to the greater diameters of cannulas for blood transfer mentioned above in this application/patent, i.e. 10 French and more than 10 French. By adjusting the number of included segments (from 4 to maximal 7) each conductive catheter/cannula part C1a to C11b may cover a range of heart H sizes. The area in the middle of the given ranges may mark the optimal, ‘safe’ choice.

The impedance Z is a value characterizing the relationship between current and voltage in AC (alternating current) circuits by dividing voltage by current. Resistance R is the real part of impedance Z. Reactance X is the imaginary part of impedance Z. Admittance Y is current divided by voltage. Conductance G is the real part of admittance Y. Susceptance B is the imaginary part admittance Y. There are simple relations between these six physical parameters. Thus, there is a choice which one of the parameters may be used for detecting the volume V.

Real time detection of volume V and/or of pressure p and/or of PV loops and/or of other parameters may be performed in less than 20 ms (milliseconds) from the respective event. Near real time detection of volume V and/or pressure p and/or of other parameters may be in less than 100 ms starting from respective event to end of signal processing.

Hemodynamic effects may have to be considered, for instance with regard to polarization. Patient security has to be considered, i.e. a current having a frequency of 60 Hz (Hertz) and/or an amplitude of 20 μA (microampere) may be capable of causing ventricular fibrillation.

Appropriate frequencies may be in the range of 2 KHz (kilohertz) to 20 KHz (Baan et. al, 1981). However, frequencies in the range of 1 kHz to 100 kHz may be used.

Constant amplitude alternating current of 0.4 mA (milliampere) (peak to peak) unipolar or bipolar may be used. The amplitude may alternatively be within a range of 0.1 mA to 1 mA. An amplitude range of 0.5 mA to 5 mA may be used especially because of larger surface area of electrodes (e.g. maintaining low current density) for larger diameters of the catheters. Increased currents may result in an improved signal to noise ratios in the detected or measured signals.

Constant currents may be used as well if appropriate. Changing of electrical parameters and/or number of used electrodes may be possible during operation and/or during setting up of the measurement arrangement.

FIG. 17 illustrates an example of hypothetical PV loops (pressure-volume) loops 1710 and 1712 within a coordinate system 1700. PV loops 1710 and 1712 may be used for instance for the adaption of a blood support circuit 106 to 1106. Coordinate system 1700 comprises a horizontal x-axis 1702 (abscissa) and a vertical y-axis 1704 (ordinate). Horizontal x-axis 1702 represents values of volume V, for instance within the range of 0 ml (milliliter) to 150 ml. Vertical y-axis 1704 represents values of pressure p within the left ventricle LV and within a range of 0 mmHg (millimeter mercury Hg column) to 150 mmHg.

A cycle of the heart-beat of heart H comprises for phases: a) isovolumetric contraction, b) systole phase, c) isovolumetric relaxation, and d) diastole phase. All phases a) to d) may occur for instance within 0.4 seconds, see arrows A1 and A2 indicating the direction of recording and/visualizing loops 1710 and 1712.

Stroke volume SV1 may be calculated from EDV1 (End Diastolic Volume)−ESV1 (End Systolic Volume). The area within the PV loops 1710 and 1712 may be a measurement for the mechanical work that is fulfilled by heart H.

Pressure loop 1710 may be measured when blood support circuitry 106 to 1106 is switched off or is operated with zero pump volume per second (0 l/min (liter per minute)). Contrary, pressure loop 1712 may be measured when blood support circuitry 106 to 1106 has a specific pump volume per minute, for instance 4 liter per minute.

As is apparent from FIG. 17, PV loop 1712 is shifted to the left and downwards if compared with the position of PV loop 1710. Furthermore, PV loop 1712 indicates a smaller stroke volume SV2 and also a smaller mechanical workload (area within loop 1712) of heart H, e.g. heart H has been unloaded and may relax. Loop 1712 has an end systolic value ESV2 which is less than EDV1. End diastolic value EDV2 of loop 1712 is less than EDV1 and also less than ESV1. A curve 1714 connects the right lower corners of loop 1710 and of loop 1712, this line may be named as EDPVR[slope] (end diastolic pressure volume relationship). This slope may be determined from a loading change.

After a while, for instance some weeks or after some month heart H may be recovered and it may be possible to load heart H again, i.e. to reduce pumping power of the pumps P1 to P11 within support circuitry 106 to 1106. This method is named as weaning. Weaning may be performed automatically up to a specific degree or fully automatically.

A line 1720 connects the upper left corners of loops 1710 and 1712. An arrow 1720 may indicate an increase in the pumping speed, e.g. an unloading of heart H.

FIG. 18 illustrates an example of a computer system 1800 which may be used for determining or measuring the volume V of a cavity, e.g. of heart H, in real time (within less than 20 ms or within less than 30 ms) or in near real time (within less than 100 ms).

Computer system 1800 may comprise:
a processor P, for instance a microprocessor or a microcontroller (may comprise more peripheral components compared to a microprocessor, e.g. digital to analog converter (DAC), analog to digital converter ADC, etc.),
a memory M, e.g. SSD (solid state device), RAM (random access memory), ROM (read only memory).

Memory M may store program data (BIOS—basic operation system), application program, and/or application data D (e.g. pressure p data D and/or volume V data D),
a bus B which connects processor P and memory M,
optional peripheral components,
an optional transmitting unit T (may transmit data D via a personal network (Bluetooth, ZigBee, etc.) or a WLAN (Wireless Local Area Network).

System 1800 may be an embedded system. Alternatively, system 1800 may comprise input/output devices, e.g. keyboard, display and/or touchscreen.

Different control schemes may be applied using for instance system 1800. One control scheme is as follows. An increase of EDV (end systolic volume), e.g. with activity, may cause an increase in pump volume to provide more unloading and circulatory support. A decrease in EDV may occur during normal activity or rest and the pump will reduce the pump volume accordingly, e.g. reduce speed or strokes per minute if a membrane pump is used. Other parameters may be used in addition to EDV or instead of EDV, for instance end diastolic pressure EDP or peek to peek pressure (Pppk).

In all embodiments one of the following methods may be used to bring or guide a guide wire and/or a catheter around or along the acute angle within the left ventricle LV. The same principles may be applied to cannula arrangement within the right ventricle RV. At least one snare may be used to catch the catheter and/or the guide wire in the left ventricle LV. The methods may be performed independent whether there is jugular access or a femoral access or another access for the catheter and/or the guide wire.

Variant A (Catching the Catheter with the Snare):
1) Introducing a catheter through the right atrium RA and through the atrial septum AS (a puncturing step may be performed earlier or using the catheter, e.g. using a needle and/or RF (radio frequency) tip/wire within the catheter). The catheter may be introduced further through the hole (puncture) in the atrial septum AS through left atrium LA, through mitral valve MV into the left ventricle LV.
2) Introducing a snare from descending aorta AO through aortic valve AV into left ventricle LV. This step may be performed also before step 1.
3) Catching the catheter in the left ventricle LV using the snare.
4) Pulling the snare and the distal end of the catheter therewith to the aorta AO.
5) Introducing a guide wire through the catheter.
6) Forwarding the guide wire out of the distal end of the catheter. Slight loosening of the snare may be optionally performed thereby.
7) As the guide wire is already within the snare, pull back the snare to a region in which only the guide wire is located but not the catheter.
8) Fix the guide wire using the snare, e.g. contract the snare and/or tighten the snare.
9) Optional, externalizing for instance the distal end of the guide wire out of body 100. This step is optionally, because the proximal end of the snare is already outside of body 100.
10) Remove catheter, e.g. pull back the catheter.
11) Introduce cannula using the guide wire, e.g. pushing the cannula along and/or over the guide wire until it is on its final place.

Variant B (Catching the Guide Wire with the Snare):
1) See step 1) of variant A.
2) Introducing a guide wire through the catheter until the distal end of the guide wire comes out of the distal end of the catheter within the left ventricle LV. The RF wire may be used also as a guide wire.
3) Introducing a snare from descending aorta AO through aortic valve AV into left ventricle LV. This step may be performed before step 1 and/or before step 2.
3) Catching the distal end of the guide wire in the left ventricle LV using the snare.
4) Fixation of the guide wire using the snare.
5) Pulling the snare and the distal end of the guide wire therewith to the aorta AO.
6) Optional, externalizing guide wire by pulling it out of body 100 using the snare. This step is optional as the snare is already outside of body 100 from where it has been introduced.
7) Remove catheter, e.g. by pulling it back along the guide wire.
8) Introduce cannula over/along the guide wire until it is on place.

Subclavian arteries/veins or other arteries/veins may be used for introducing the snare(s) because the snares require smaller diameters, e.g. less than 10 French (1 French equal to ⅓ mm (millimeter)) or less than 8 French, e.g. more than 3 French, compared to the diameters of the cannula(s).

In the following, details of a method for puncturing trans-septal through the atrial septum AS of the heart H are provided. However, other methods may be used as well, for instance using a needle. A catheter and/or a wire may be used which has a distal tip which can be heated, for instance using RF (radio frequency) energy, alternating current (ac), direct current (dc) etc. Thus, e.g. a hole may be burned into the septum, e.g. the atrial septum AS, during puncturing, for instance using temperatures above 100° C. (degrees Celsius) or above 200° C., less than 1000° C. for instance.

The RF (radio frequency) may be in the range of 100 kHz (kilohertz) to 1 MHz (Megahertz) or in the range of 300 kHz to 600 kHz, for instance around 500 MHz, i.e. in the range of 450 kHz to 550 kHz, e.g. 468 kHz. The power of the radio frequency energy may have a maximum of 50 Watt. A power range of 5 W (watt) to 100 W may be used, for instance a range of 10 W to 50 W. A sinus current/voltage may be used for the RF. The sinus current/voltage may be continuous. Alternatively, a pulsed sinus current/voltage may be used for the RF.

All parameters or some of the parameters of the RF equipment may be adjustable by an operator who performs the puncturing, for instance dependent on the specifics of the septum, e.g. normal septum, fibrotic septum, aneurysmal septum, etc. Preferably, the power may be adjustable.

A solution of Baylis Medical (may be a trademark), Montreal, Canada may be used, for instance NRG® trans-septal needle or Supra Cross® RF Wire technology. RF generator of type RFP-100A or a further development of this model may be used. This RF generator uses for example a frequency of 468 kHz (kilohertz).

A single puncture of the septum may be performed from a jugular access or from a femoral access or from another appropriate access using the RF energy. Smaller angles may be possible for the catheter if for instance compared with a needle.

Puncturing of the atrial septum may be assisted by at least one medical imaging method, preferably by at least two medical imaging methods. US (ultra-sonic) echo imaging may be used to visualize the movement of heart H and the location of the valves of heart H. No dangerous radiation may result from ultra-sonic imaging. An ultra-sonic transmitter may be introduced for instance via the esophagus, e.g. trans esophagus echo (TEE) may be used. X-ray radiation preferably in combination with fluorescence (fluoroscopy), may be used in order to visualize the location of catheters (comprising for instance at least one X-ray marker, or the devises are usually radiopaque) and/or the location of guide wire(s), snares etc.

Thus, transseptal puncturing or puncturing of other tissue may be guided by TEE and by fluoroscopy or by other imaging methods. At least two different image generating methods may be used.

In all embodiments mentioned above, it is also possible to use a soft guide wire and a stiffer guide wire which does not bend so easy if compared with the soft guide wire. The following steps may be performed, preferably in combination with snaring:
1) Introduce a soft guide wire.
2) Introduce catheter using the soft wire as a guide.
3) Optionally, remove soft wire, for instance by pulling back the soft wire out of the catheter.
4) Introduce stiffer guide wire into the catheter, e.g. there may be a change of wire from soft wire to the stiffer wire.

The catheter may be removed, e.g. pulled back. Thereafter, the stiffer wire may be used to introduce a cannula or cannulas.

Cannula tips may be used within the cage arrangements. Alternatively, tips without cage arrangements may be used. The tip may have only one end hole. Alternatively the tip may comprise only radial holes. Alternatively, the tip may comprise an end hole and radial holes. In all three cases electrodes and/or pressure sensors may be arranged at the tip.

A catheter/cannula may also be inserted transseptal through the ventricle septum. Although, several conductance cannula/catheter portions are used in several of the embodiments mentioned above, it is of course possible to use cannulas/catheters comprising less or more conductance cannula/catheter portions than illustrated for instance in a respective on of the FIGS. 1 to 11. Thus, one or two or more than three conductance cannula/catheter portions may be omitted thereby still using the proposed invention.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure.

Moreover, the scope of the present application is not intended to be limited to the particular embodiments of the system, process, manufacture, method or steps described in the present disclosure. As one of ordinary skill in the art will readily appreciate from the disclosure of the present disclosure systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. It is possible to combine embodiments mentioned in the first part with each other or to combine embodiments mentioned in the description of the Figures with each other. Further, it is possible to combine embodiments mentioned in the first part of the description with examples of the second part of the description which relates to FIGS. 1 to 18.

1. Cannula (110 to 1110, CA1500, CA1600) for detection of the volume (V) of a cavity within a body (100), comprising:
a lumen portion (LP) comprising and inner surface (ISF) and an outer surface (OSF),
a first opening (OP1) on a first end (FE) of the lumen portion (LP),
a second opening (OP2) on a second end (SE) of the lumen portion (LP),
wherein the first opening (OP1) and the second opening (OP2) have an inner diameter that allows endovascular blood circuit support of the blood circuit of a subject (Pat),
a lumen (LU) extending from the first end (FE) to the second end (SE) within the lumen portion (LP), and at least one series (SER1) of electrodes (EE1 to EE2) comprising at least two electrodes (EE1 to EE2),
wherein the series of electrodes (EE1 to EE2) is arranged along at least a part of the lumen portion (LP) at the outer surface (OSF),
wherein the at least one series (SER1) of electrodes (EE1 to EE2) is configured to be used for the detection of the amount of a volume (V) of a cavity within a body (100) of the subject (Pat).

2. Cannula (110 to 1110, CA1500, CA1600) according to claim 1, wherein the length from the first end (FE) to the second end (SE) is at least 50 centimeters or at least 70 centimeters at least 90 centimeters, and
wherein the outer diameter of the lumen portion (LP) is at least 10 French or at least 15 French or at least 20 French or at least 25 French or at least 30 French.

3. Cannula (110 to 1110, CA1500, CA1600) according to claim 1 or 2 wherein the number of electrodes (EE1 to EE2) is at least 5 or at least 6 or at least 15 or at least 25.

4. Cannula (110 to 1110, CA1500, CA1600) according to any one of the preceding claims, comprising at least one pressure sensor (PS1 to PS6) or at least two pressure sensors (PS1 to PS6) or at least three pressure sensors (PS1 to PS6) or at least four pressure sensors (PS1 to PS6),
wherein the pressure sensor (PS1 to PS6) is configured or the pressure sensors (PS1 to PS6) are configured to detect the pressure within at least one cavity of the body (100).

5. Cannula (110 to 1110, CA1500, CA1600) according to any one of the preceding claims, wherein the cannula comprises a second series (SER2) of the at least one series (SER1, SER2) of electrodes (EE1 to EE2),
wherein the first series (SER1) is arranged closer to the second end (SE) than the second series (SER2),
wherein there is a group distance (GD1) between a last electrode (EE2) of the first series (SER1) which is farthest away from the second end (SE) and a first electrode (EE1a) of the second series (SER2) which is closest to the second end (SE), and
wherein the group distance (GD) is greater than or greater than twice or greater than the threefold of the greatest distance between two adjacent electrodes of the first series (SER1) and/or greater than the greatest distance between two adjacent electrodes of the second series (SER2).

6. Cannula (110 to 1110, CA1500, CA1600) according to claim 5, wherein the electrodes (EE1 to EE2) of the first series (SER1) are configured to determine the volume (V) of the left ventricle (LV) of a heart (H) and wherein the second series (SER2) of electrodes (EE1a to EE2a) is configured to determine the volume (V) of the left atrium (LA) of the heart (H).

7. Cannula (110 to 1110, CA1500, CA1600) according to any one of the claims 1 to 4, wherein the cannula comprises a second series (SER2) of the at least one series (SER1, SER2) of electrodes (EE1 to EE2),
wherein the first series (SER1) is arranged closer to the second end (SE) than the second series (SER2),
wherein there is a group distance (GD2) between a last electrode (EE2) of the first series (SER1) which is farthest away from the second end (SE) and a first electrode (EE1a) of the second series (SER2) which is closest to the second end (SE), and
wherein the group distance (GD2) is less than the threefold or less than twice the greatest distance or less the greatest distance between two adjacent electrodes (EE1 to EE2) of the first series (SER1) and greater than the greatest distance between two adjacent electrodes (EE1a to EE2a) of the second series (SER2).

8. Cannula (110 to 1110, CA1500, CA1600) according to claim 7, wherein the electrodes (EE1 to EE2) of the first series (SER1) of electrodes are configured to determine the volume (V) of the right ventricle (RV) of a heart (H) and wherein the second series (SER2) of electrodes (EE1a to EE2a) is configured to determine the volume (V) of the right atrium (RA) of the heart (H).

9. Cannula (110 to 1110, CA1500, CA1600) according to any one of the preceding claims, wherein there is at least one radial hole (252) or wherein there are at least two radial holes (252) or at least three radial holes between the electrodes (EE1 to EE2) of the at least one series (SER1, SER2) of electrodes.

10. Cannula (110 to 1110, CA1500, CA1600) according to any one of the preceding claims, wherein the cannula is a single lumen cannula (110, 410, 510, 540, 610, 640, 710, 740, 810, 840, 910, 940).

11. Cannula (110 to 1110, CA1500, CA1600) according to claim 10, wherein the cannula is bidirectional cannula comprising at least one third opening between the first opening and the second opening,
wherein the cannula comprises at least one valve arrangement which is configured to enable flows between different openings of the cannula depending on the direction of the flow within a common portion of the cannula.

12. Cannula (110 to 1110, CA1500, CA1600) according to any one of the claims 1 to 9, wherein the cannula is an inner cannula (I1 to I3) of a dual lumen cannula (210, 310, 1010, 1040, 1110),
wherein the dual lumen cannula (210, 310, 1010, 1040, 1110) comprises an outer cannula (O1 to O3),
wherein the inner cannula (I1 to I3) is arranged within the outer cannula (O1 to O3) and extends more distally than the outer cannula (O1 to O3).

13. Cannula (110 to 1110, CA1500, CA1600) according to any one of the claims 1 to 9, wherein the cannula is an outer cannula (O1 to O3) of a dual lumen cannula (210, 310, 1010, 1040, 1110),
wherein the dual lumen cannula (210, 310, 1010, 1040, 1110) comprises an inner cannula (I1 to I3) arranged within the outer cannula (O1 to O3) and extending more proximally than the outer cannula (O1 to O3).

14. Cannula (110 to 1110, CA1500, CA1600) according to claim 12 or 13 wherein the inner cannula (I1 to 13) is axially movable (M1, M2) with regard to the outer cannula (O1 to O3), preferably along a distance which is at least half the length, the length or more than the length of the outer cannula (O1 to O3).

15. Cannula (110 to 1110, CA1500, CA1600) according to any one of the preceding claims, comprising at least one volume expandable arrangement (116),
wherein the volume expandable arrangement (116) defines a first volume in a non-expanded state,
wherein the volume expandable arrangement (116) defines a second volume in an expanded state,
and wherein the second volume is greater than the first volume by at least factor 2, at least factor 3 or by at least factor 4.

16. Cannula (110 to 1110, CA1500, CA1600) according to any one of the preceding claims, comprising at least one cage arrangement (116, 216, 316, 416 to 1146) and/or at least one membrane (648, 919, 949, 1049, 1089, 1149), arranged at at least one wire (118 to 618, 918, 948 1048, 1088, 1148) of the cage arrangement (116, 216, 316, 416 to 1146).

17. Cannula (110 to 1110, CA1500, CA1600) according to any one of the preceding claims, wherein the cannula (110 to 1110, CA1500, CA1600) comprises a flexible material extending from the first end (FE) to the second end (SE).

18. Set, comprising a cannula (110 to 1110, CA1500, CA1600) according to one of the preceding claims, and
an extracorporeal pump (P1 to P5, P9 to P11).

19. Set according to claim 18, comprising an electronic detection unit (1604).

20. Method for determining the volume (V) of a cavity in a living body (100), comprising:
determining a volume (V) and/or a pressure (p) in a cavity of a living body (100) and generating corresponding data (D) using at least one cannula (110 to 1110, CA1500, CA1600) according to one of the claims 1 to 17 or a set according to claim 18 or 19,
preferably storing the generated data (D),
evaluating the generated data (D) or the stored data, and
changing at least one parameter of an extracorporeal or an intracorporeal blood pump (P1 to P5, P9 to P11) which pumps blood through the cannula (110 to 1110, CA1500, CA1600).

21. Method according to claim 10, comprising:
determining volume (V) and/or a pressure (p) in at least two chambers of the heart (H) using two different portions (C3a, C3b) of the cannula (110 to 1110, CA1500, CA1600) or two of the cannulas (110 to 1110, CA1500, CA1600), or
determining volume (V) and/or pressure (p) in at least three chambers of the heart (H) using the cannula (110 to 1110, CA1500, CA1600) or using at least two of the cannulas (110 to 1110, CA1500, CA1600), or
determining volume (V) and/or pressure (p) in at least four chambers of the heart (H) using at two of the cannulas (1010, 1040).

22. Method according to claim 20 or 21, wherein the method is used for weaning a patient (Pat) from a blood circuit support system (106, 206, 306 to 1106).

23. Computer program product (M) comprising instructions that are executable to cause a processor (P) to perform the generation of data (D) and/or the evaluation according to the method of any one of the claims 20 to 22.

24. Computer system (1800), comprising a computer program product (M) according to claim 23, wherein the computer system (1800) is configured to perform the generation of data (D) and/or the evaluation according to the method of any one of the claims 20 to 22.
